(19) United States
(12) Patent Application Publication
(10) Pub. No.: US 2002/0049191 A1
(43) Pub. Date: Apr. 25, 2002

(54) Cephalosporin derivatives

(76) Inventors: Dane M. Springer, North Haven, CT (US); Bing Y. Luh, Killingworth, CT (US); Stanley V. D'Andrea, Wallingford, CT (US); Thomas W. Hudyma, Durham, CT (US); Oak K. Kim, Milford, CT (US)

Correspondence Address: MARLA J MATHIAS, BRISTOL-MYERS SQUIBB COMPANY, PATENT DEPARTMENT, P O BOX 4000, PRINCETON, NJ, 08543-4000, US

(21) Appl. No.: 09/867,225

(22) Filed: May 29, 2001

Related U.S. Application Data
(63) Continuation-in-part of application No. 09/309,955, filed on May 11, 1999, now abandoned.
Application No. 09/309,955 is a continuation-in-part of application No. 08/972,869, filed on Nov. 18, 1997, now abandoned.
(60) Provisional application No. 60/034,046, filed on Nov. 27, 1996.

(51) Int. Cl. A61K 31/675; A61K 31/545; C07D 501/14

(52) U.S. Cl. 514/203; 514/79; 540/224

(57) ABSTRACT

The present invention provides a novel series of cephem derivatives of the general formula I

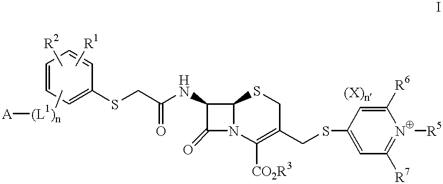

wherein R¹, R², R³, R⁵, R⁶, R⁷, A, L¹, X, n and n′ are as defined herein above. The compounds of formula I are antibacterial agents useful in the treatment of infections in humans and other animals caused by a variety of gram-positive bacteria, particularly methicillin-resistant Staphylococcus aureus. Also included in the invention are processes for preparing the compounds of formula I and pharmaceutical compositions containing said compounds in combination with pharmaceutically acceptable carriers, diluents or excipients.

### CROSS-REFERENCE TO RELATED APPLICATIONS

This application is a continuation-in-part of our co-pending application Ser. No. 09/309,955 filed May 11, 1999 which is a continuation-in-part of application Ser. No. 08/972,869 filed Nov. 18, 1997, now abandoned, which claims the priority of provisional application serial No. 60/034,046 filed Nov. 27, 1996.

### BACKGROUND OF THE INVENTION

1. Field of the Invention

The present invention is directed to new cephem derivatives represented by the general formula I

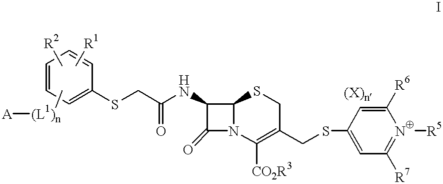

wherein R¹ is hydrogen or halogen; R² is halogen; R³ is hydrogen or a negative charge; A is CO₂R⁴, PO₃(R⁴)₂, SO₃R⁴ or tetrazolyl; R⁴ at each occurrence is either hydrogen or a negative charge; L¹ is a furanyl group, a thienyl group, a C₂-C₁₀ alkylene group, or a C₂-C₁₀ alkylene group wherein one or more of the carbon atoms of said C₂-C₁₀ alkylene may be replaced by S, SO, SO₂, SO₂NH, C(O)NH or NHC(O) and wherein there may be one or more double bonds between adjacent carbon atoms; and n is 0 or 1; R⁵ is selected from the group consisting of hydrogen, NH₂, pyrrolidinyl, N═CHR¹⁰, C₃-C₆ cycloalkyl, C₁-C₆ alkyl, substituted C₂-C₆ alkyl, phenyl, and substituted phenyl, wherein said substituted C₂-C₆ alkyl is a C₂-C₆ alkyl substituted by one or more substituents each independently selected from the group consisting of OH, NR⁸R⁹, NR⁸R⁹R^{9′}, 4-morpholinyl, 4-morpholinyl quaternized by a C₁-C₆ alkyl group, oxo, halogen, CO₂R⁴, SO₃R⁴, PO₃(R⁴)₂, 3-imidazolium, 3-imidazolium substituted by one to two C₁-C₆ alkyl groups, tetrazolyl, and tetrazolyl substituted by one to two C₁-C₆ alkyl groups, and wherein said substituted phenyl is a phenyl substituted by one to three substituents each independently selected from the group consisting of OH, NR⁸R⁹, CO₂R⁴, SO₃R⁴, PO₃(R⁴)₂, 4-morpholinyl, 4-morpholinyl quaternized by a C₁-C₆ alkyl, imidazolyl, imidazolyl substituted by one to two C₁-C₆ alkyl groups, tetrazolyl, and tetrazolyl substituted by one to two C₁-C₆ alkyl groups; R⁶ and R⁷ are each independently hydrogen or C₁-C₆ alkyl; R⁸, R⁹ and R^{9′} are each independently hydrogen or C₁-C₆ alkyl; R¹⁰ is furanyl or thienyl wherein said furanyl or thienyl is optionally substituted by —CO₂R⁴ or —SO₃R⁴; X at each occurrence is a counterion; and n′ is 0 to 2. The derivatives are antibacterial agents useful against gram-positive bacteria and especially useful in the treatment of diseases caused by methicillin-resistant Staphylococcus aureus (also referred to below as MRSA or methicillin-resistant S. aureus).

2. Description of the Prior Art

The literature discloses a vast number of cephem derivatives having a wide variety of C-3 and C-7 substituents.

With respect to the C-7 substituents of the present invention, U.S. Pat. No. 3,345,366 discloses cephem derivatives of the type

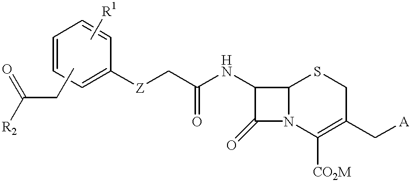

wherein R₁ is hydrogen or chloro, R₂ is hydroxy or amino, Z is oxygen or sulfur, A is acetoxy or N-pyridinium and M is hydrogen, pharmaceutically acceptable non-toxic cations or an anionic charge when A is N-pyridinium. This approach was also discussed in Antimicrob. Ag. Chemother. Meeting, 1968, pgs 109-114 (Hobby, G. L.) and JP 50083383.

EP 638,574 A1 discloses cephem derivatives of the general formula

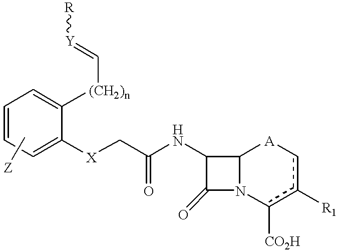

wherein

X is absent, O, S, SO, SO₂, or NH;

Y is CH, or N;

Z is H, halogen, OH, C₁-C₅ O-alkyl, —OCH₂CONH₂, —OCONH₂, —OSO₂NH₂, —OCH₂CN, —NH₂ either as such or substituted with C₁-C₆ alkyl radicals, —NHCOCH₃, —NHSO₂CH₃, —NHSO₂(4-methylphenyl), amides of C₁-C₄ linear acids, amides of benzene and toluene derivatives, —NO₂, —NO, —CHO, —CH₂OH, —CO₂H, —SH, —SOH, —SO₂H, —SO₃H, —S—C₁-C₃alkyl, or —CF₃;

R is H, OH, C₁-C₅-O-alkyl with the alkyl residue possibly containing halogens, acid functionalities either free or salified with alkaline or alkaline earth metals, basic functions such as —OCH₂CH₂NH₂, —OCH₂CH₂NHCH₃, —OCH₂(o, m, p)-pyridinyl, —OCH₂CN, —OCH₂CONH₂, —OCH₂SO₂NH₂;

n is0to 4;

A is S, O, CH₂, SO, or SO₂;

R₁ is a structural group characteristic of cephalosporins such as Cl, H, OCH₃, CH₂OCH₃, CH₃, —CH═CH-CH₃, CF₃, —CO₂R₂, —SO₂R where R₂ is an alkyl or aryl radical, —CH₂OCOCH₃, CH═CH₂,

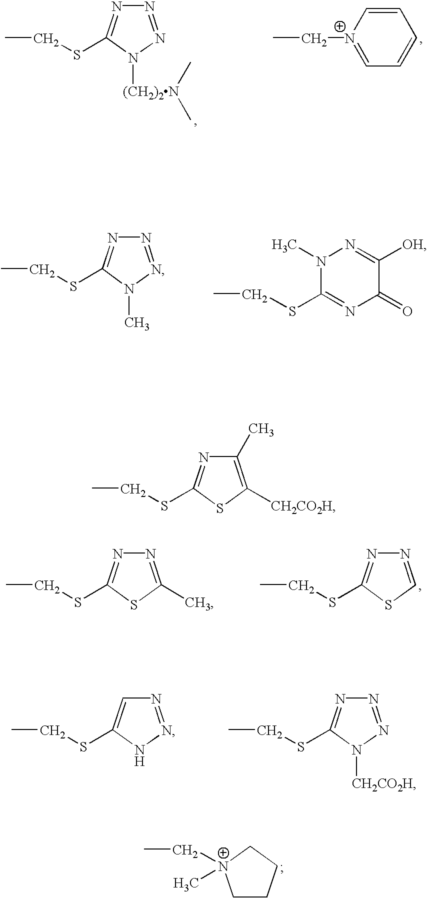

their pharmaceutically acceptable salts and their C6 and C7 epimers.

The C-3 substituents employed in the compounds of the present invention are known in the cephem art, but have not previously been combined with the C-7 substituents of the present invention. Applicants have discovered that the combination of C-3 and C-7 substituents provided in the compounds of the present invention unexpectedly gives the desired solubility, activity and toxicity profile needed for commercially viable anti-MRSA cephem products.

### SUMMARY OF THE INVENTION

The present invention provides a novel series of cephem derivatives of the general formula I

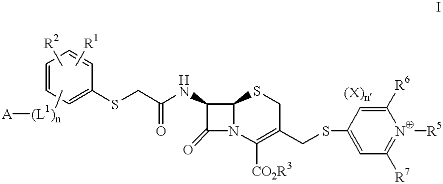

wherein R¹ is hydrogen or halogen; R² is halogen; R³ is hydrogen or a negative charge; A is CO₂R⁴, PO₃(R⁴)₂, SO₃R⁴ or tetrazolyl; R⁴ at each occurrence is either hydrogen or a negative charge; L¹ is a furanyl group, a thienyl group, a C₂-C₁₀ alkylene group, or a C₂-C₁₀ alkylene group wherein one or more of the carbon atoms of said C₂-C₁₀ alkylene may be replaced by S, SO, SO₂, SO₂NH, C(O)NH or NHC(O) and wherein there may be one or more double bonds between adjacent carbon atoms; and n is 0 or 1; R⁵ is selected from the group consisting of hydrogen, NH₂, pyrrolidinyl, N═CHR¹⁰, C₃-C₆ cycloalkyl, C₁-C₆ alkyl, substituted C₂-C₆ alkyl, phenyl, and substituted phenyl, wherein said substituted C₂-C₆ alkyl is a C₂-C₆ alkyl substituted by one or more substituents each independently selected from the group consisting of OH, NR⁸R⁹, NR⁸R⁹R^{9′}, 4-morpholinyl, 4-morpholinyl quaternized by a C₁-C₆ alkyl group, oxo, halogen, CO₂R⁴, SO₃R⁴, PO₃(R⁴)₂, 3-imidazolium, 3-imidazolium substituted by one to two C₁-C₆ alkyl groups, tetrazolyl, and tetrazolyl substituted by one to two C₁-C₆ alkyl groups, and wherein said substituted phenyl is a phenyl substituted by one to three substituents each independently selected from the group consisting of OH, NR⁸R⁹, CO₂R⁴, SO₃R⁴, PO₃(R⁴)₂, 4-morpholinyl, 4-morpholinyl quaternized by a C₁-C₆ alkyl, imidazolyl, imidazolyl substituted by one to two C₁-C₆ alkyl groups, tetrazolyl, and tetrazolyl substituted by one to two C₁-C₆ alkyl groups; R⁶ and R⁷ are each independently hydrogen or C₁-C₆ alkyl; R⁸, R⁹ and R^{9′} are each independently hydrogen or C₁-C₆ alkyl; R¹⁰ is furanyl or thienyl wherein said furanyl or thienyl is optionally substituted by —CO₂R⁴ or —SO₃R⁴; X at each occurrence is a counterion; and n′ is 0 to 2.

The compounds of formula I are antibacterial agents useful in the treatment of infections in humans and other animals caused by a variety of gram-positive bacteria, particularly methicillin-resistant S. aureus.

Also included in the invention are processes for preparing the compounds of formula I and pharmaceutical compositions containing said compounds in combination with pharmaceutically acceptable carriers, diluents or excipients.

### DETAILED DESCRIPTION

The present invention provides novel cephem derivatives of general formula I above which are antibacterial agents useful in the treatment of infectious diseases in humans and other animals. The compounds exhibit good activity against a wide variety of gram-positive microorganisms, e.g. S. pneumoniae, S. pyogenes, S. aureus, E. faecalis, S. epidermidis and S. hemolyticus, and are particularly useful against strains of methicillin-resistant S. aureus.

The compounds of formula I are characterized by a pyridinium thiomethyl group of the type

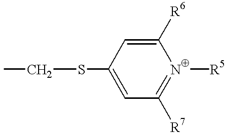

at the 3-position of the cephem ring and a 7-substituent of the type

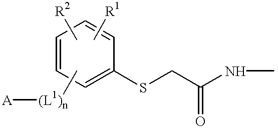

wherein R¹ is hydrogen or halogen; R² is halogen; A is CO₂R⁴, PO₃(R⁴)₂, SO₃R⁴ or tetrazolyl; R⁴ at each occurrence is either hydrogen or a negative charge; L¹ is a furanyl group, a thienyl group, a C₂-C₁₀ alkylene group, or a C₂-C₁₀ alkylene group wherein one or more of the carbon atoms of said C₂-C₁₀ alkylene may be replaced by S, SO, SO₂, SO₂NH, C(O)NH or NHC(O) and wherein there may be one or more double bonds between adjacent carbon atoms; n is 0 or 1; R⁵ is selected from the group consisting of hydrogen, NH₂, pyrrolidinyl, N═CHR¹⁰, C₃-C₆ cycloalkyl, C₁-C₆ alkyl, substituted C₂-C₆ alkyl, phenyl, and substituted phenyl, wherein said substituted C₂-C₆ alkyl is a C₂-C₆ alkyl substituted by one or more substituents each independently selected from the group consisting of OH, NR⁸R⁹,NR⁸R⁹R^{9′}, 4-morpholinyl, 4-morpholinyl quaternized by a C₁-C₆ alkyl group, oxo, halogen, CO₂R⁴, SO₃R⁴, PO₃(R⁴)₂, 3-imidazolium, 3-imidazolium substituted by one to two C₁-C₆ alkyl groups, tetrazolyl, and tetrazolyl substituted by one to two C₁-C₆ alkyl groups, and wherein said substituted phenyl is a phenyl substituted by one to three substituents each independently selected from the group consisting of OH, NR⁸R⁹, CO₂R⁴, SO₃R⁴, PO₃(R⁴)₂, 4-morpholinyl, 4-morpholinyl quaternized by a C₁-C₆ alkyl, imidazolyl, imidazolyl substituted by one to two C₁-C₆ alkyl groups, tetrazolyl, and tetrazolyl substituted by one to two C₁-C₆ alkyl groups; R⁶ and R⁷ are each independently hydrogen or C₁-C₆ alkyl; R⁸, R⁹ and R^{9′} are each independently hydrogen or C₁-C₆ alkyl; R¹⁰ is furanyl or thienyl wherein said furanyl or thienyl is optionally substituted by —CO₂R⁴ or —SO₃R⁴; X at each occurrence is a counterion; and n′ is 0 to 2.

A preferred embodiment of the present invention comprises a compound of the formula IA

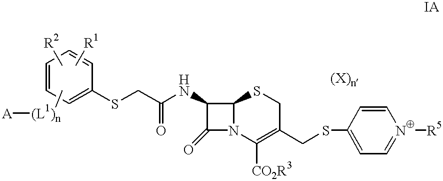

wherein R¹ is hydrogen or halogen; R² is halogen; R³ is hydrogen or a negative charge; A is CO₂R⁴, PO₃(R⁴)₂, SO₃R⁴ or tetrazolyl; R⁴ at each occurrence is hydrogen or a negative charge; L¹ is a furanyl group, a thienyl group, a C₂-C₁₀ alkylene group, or a C₂-C₁₀ alkylene group wherein one or more of the carbon atoms of said C₂-C₁₀ alkylene may be replaced by S, SO, SO₂, SO₂NH, C(O)NH or NHC(O) and wherein there may be one or more double bonds between adjacent carbon atoms; n is 0 or 1; R⁵ is selected from the group consisting of hydrogen, NH₂, pyrrolidinyl, N═CHR¹⁰, C₃-C₆ cycloalkyl, C₁-C₆ alkyl, substituted C₂-C₆ alkyl, phenyl, and substituted phenyl, wherein said substituted C₂-C₆ alkyl is a C₂-C₆ alkyl substituted by one or more substituents each independently selected from the group consisting of OH, NR⁸R⁹, NR⁸R⁹R^{9′}, 4-morpholinyl, 4-morpholinyl quaternized by a C₁-C₆ alkyl group, oxo, halogen, CO₂R⁴, SO₃R⁴, PO₃(R⁴)₂, 3-imidazolium, 3-imidazolium substituted by one to two C₁-C₆ alkyl groups, tetrazolyl, and tetrazolyl substituted by one to two C₁-C₆ alkyl groups, and wherein said substituted phenyl is a phenyl substituted by one to three substituents each independently selected from the group consisting of OH, NR⁸R⁹, CO₂R⁴, SO₃R⁴, PO₃(R⁴)₂, 4-morpholinyl, 4-morpholinyl quaternized by a C₁-C₆ alkyl, imidazolyl, imidazolyl substituted by one to two C₁-C₆ alkyl groups, tetrazolyl, and tetrazolyl substituted by one to two C₁-C₆ alkyl groups; R⁸, R⁹ and R^{9′} are each independently hydrogen or C₁-C₆ alkyl; R¹⁰ is furanyl or thienyl wherein said furanyl or thienyl is optionally substituted by —CO₂R⁴ or —SO₃R⁴; X at each occurrence is a counterion; and n′ is 0 to 2.

Another preferred embodiment comprises a compound of the formula IB

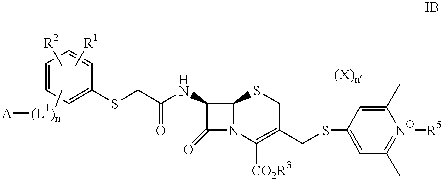

wherein R¹ is hydrogen or halogen; R² is halogen; R³ is hydrogen or a negative charge; A is CO₂R⁴, PO₃(R⁴)₂, SO₃R⁴ or tetrazolyl; R⁴ at each occurrence is hydrogen or a negative charge; L¹ is a furanyl group, a thienyl group, a C₂-C₁₀ alkylene group, or a C₂-C₁₀ alkylene group wherein one or more of the carbon atoms of said C₂-C₁₀ alkylene may be replaced by S, SO, SO₂, SO₂NH, C(O)NH or NHC(O) and wherein there may be one or more double bonds between adjacent carbon atoms; n is 0 or 1; R⁵ is selected from the group consisting of hydrogen, NH₂, pyrrolidinyl, N═CHR¹⁰, C₃-C₆ cycloalkyl, C₁-C₆ alkyl, substituted C₂-C₆ alkyl, phenyl, and substituted phenyl, wherein said substituted C₂-C₆ alkyl is a C₂-C₆ alkyl substituted by one or more substituents each independently selected from the group consisting of OH, NR⁸R⁹, NR⁸R⁹R^{9′}, 4-morpholinyl, 4-morpholinyl quaternized by a C₁-C₆ alkyl group, oxo, halogen, CO₂R⁴, SO₃R⁴, PO₃(R⁴)₂, 3-imidazolium, 3-imidazolium substituted by one to two C₁-C₆ alkyl groups, tetrazolyl, and tetrazolyl substituted by one to two C₁-C₆ alkyl groups, and wherein said substituted phenyl is a phenyl substituted by one to three substituents each independently selected from the group consisting of OH, NR⁸R⁹, CO₂R⁴, SO₃R⁴, PO₃(R⁴)₂, 4-morpholinyl, 4-morpholinyl quaternized by a C₁-C₆ alkyl, imidazolyl, imidazolyl substituted by one to two C₁-C₆ alkyl groups, tetrazolyl, and tetrazolyl substituted by one to two C₁-C₆ alkyl groups; R⁸, R⁹ and R^{9′} are each independently hydrogen or C₁-C₆ alkyl; R¹⁰ is furanyl or thienyl wherein said furanyl or thienyl is optionally substituted by —CO₂R⁴ or —SO₃R⁴; X at each occurrence is a counterion; and n′ is 0 to 2.

Particularly preferred groups of the formula

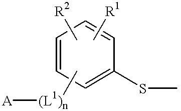

in the above-described compounds of formula I, IA and IB include but are not limited to

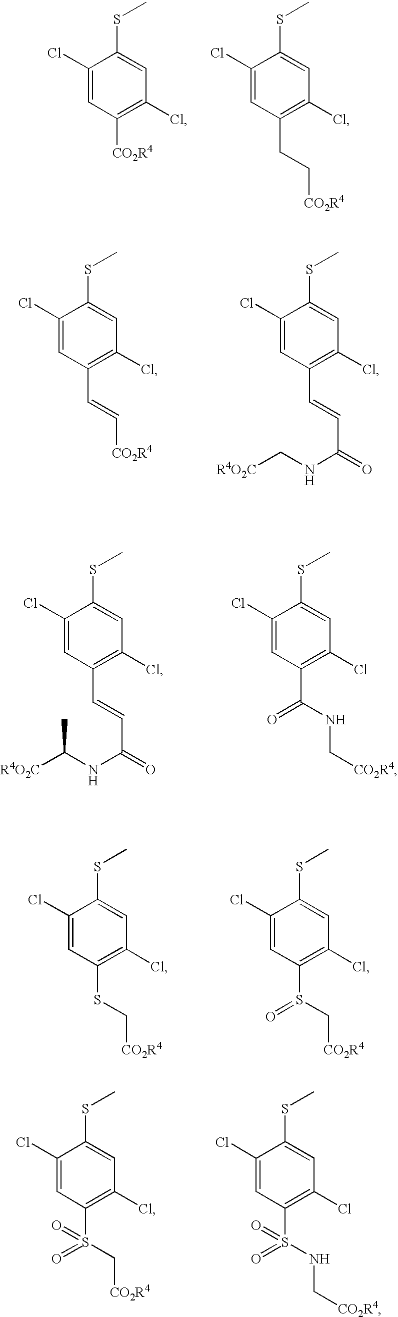

Particularly preferred groups of the formula

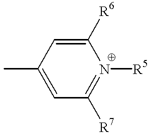

include but are not limited to

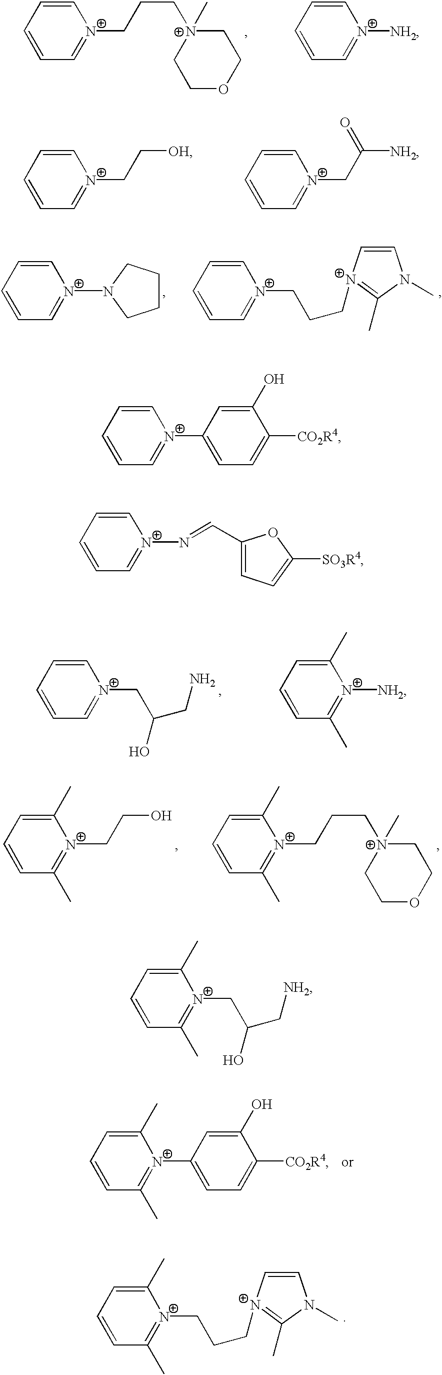

Specific preferred embodiments of the present invention are depicted below as (a) through (v). It is understood by one skilled in the art that the counterions, when depicted for embodiments (a) through (v), provide a nonlimiting example and that other counterions may be similarly employed and that other salt forms are possible.

[chemical structure]

To elaborate on the definitions for substituents of the formula I, IA and IB compounds:

(a) The term “halogen” includes chloro, bromo, fluoro and iodo, and is preferably chloro or bromo and most preferably chloro.

(b) The term “alkyl” groups may be straight or branched chains containing the specified number of carbon atoms. For example, the term “C₁-C₆ alkyl” is an alkyl group containing one to six carbon atoms and includes groups such as methyl, ethyl, propyl, isopropyl, butyl, isobutyl, tertiary-butyl, 4-methylbutyl, pentyl, hexyl and the like.

The term “counterion” as used herein is intended to include both counter anions and counter cations. The counterion, when present in a compound of Formula I, is selected such that the net charge of a compound of Formula I is zero, i.e. the compound as a whole is neutral. The counter anions are those anions obtained upon deprotonation of nontoxic inorganic or organic acids, e.g. counter anions obtained by deprotonation of acids such as hydrochloric, phosphoric, sulfuric, maleic, acetic, citric, succinic, benzoic, fumaric, mandelic, p-toluenesulfonic, methanesulfonic, ascorbic, lactic, gluconic, trifluoracetic, hydroiodic, hydrobromic, and the like. For example, the counter anions obtained from hydrochloric acid, hydrobromic acid, and acetic acid are chloride, bromide, and acetate, respectively.

Some of the compounds of the present invention may have an acidic hydrogen and can, therefore, be converted with bases in a conventional manner into pharmaceutically acceptable salts which contain counter cations. Such salts, may posess counter cations such as, for example, ammonium, alkali metal salts, particularly sodium or potassium, alkaline earth metal salts, particularly calcium or magnesium. Pharmaceutically acceptable salts may also be formed with suitable organic bases such as lower alkylamines (methylamine, ethylamine, cyclohexylamine, and the like) or with substituted lower alkylamines (e.g. hydroxyl-substituted alkylamines such as diethanolamine, triethanolamine or tris-(hydroxymethyl)amino-methane), or with bases such as piperidine or morpholine. In these instances the counter cation is the protonated organic base. For example the counter cations generated by protonation of methylamine and ethylamine are methylammonium and ethylammonium, respectively.

It is understood that compounds of the present invention may exist in various salt forms. Scheme 1, below, depicts some of the salt forms possible for a compound of Formula I when the group represented by R⁵ is a neutral nonionizable group. Formula Ic in Scheme 1 depicts the compound as a zwitterion/mono salt form wherein the acid group, A, has a counter cation. Formula Id depicts the compound in zwitterionic form, with the group A as the free acid. Formula Ie shows the compound as a mono salt form wherein group A exists as the free acid and the quaternary nitrogen of the thiopyridinium moiety has a counter anion. As shown in Scheme 1 the various forms may be interconverted by treatment with a suitable acid or base. For example, treatment of the zwitterionic compound of Formula Id with one or more equivalents of a suitable base can provide the zwitterion, mono salt of Formula Ic. Likewise, treatment of the zwitterionic compound of Formula Id with one or more equivalents of a suitable acid can provide the mono salt form of Formula Ie. It is to be understood that a compound of formula I wherein the group represented by R⁵ is a neutral nonionizable group may exist as a mixture of the various salt forms depicted in Scheme 1.

[chemical structure]

Scheme 2, below, depicts some of the salt forms possible for compounds of Formula I when the group represented by R⁵ contains a quaternary nitrogen moiety. The quaternary nitrogen moiety within R⁵ could be, for example, part of a nitrogen linked quaternary imidazole, morpholine or amino group (i.e. a nitrogen linked imidazolium, morpholinium or ammonium group). The compound of Formula If in Scheme 2 depicts the compound as a bis zwitterion wherein the counter anions for both the quaternary nitrogen of the thiopyridinum and R¹ group are internal (i.e. the C-4 carboxylate and the anionic group A). Treatment of a compound of formula If with one equivalent of an appropriate acid, HX, can result in formation of mono zwitterion, mono salt form of formula Ig, wherein the group A exists as the free acid. Treatment of the mono zwitterion, mono salt form of formula Ig with an additional one or more equivalents of an appropriate acid, HX, can result in formation of the bis salt form of formula Ih. Likewise, the bis salt compound of formula Ih can be treated with one equivalent of an appropriate base to regenerate the mono zwitterion, mono salt form of formula Ig which can then be treated with an additional one or more equivalents of appropriate base to form the bis zwitterion compound of formula If. It is to be understood that a compound of formula I wherein the group represented by R⁵ contains a quaternary nitrogen moiety may exist as a mixture of the various salt forms as depicted in Scheme 2.

[chemical structure]

Scheme 3, below, depicts some of the salt forms possible for compounds of Formula I when the group represented by R⁵ contains an ionizable acidic moiety such as a carboxylic acid or sulfonic acid group. The compound of formula Ii depicts the compound as an acid salt form wherein the counterion is a counter anion. Treatment of the compound of formula Ii with one equivalent of an appropriate base can provide the zwitterionic compound of formula Ij. Treatment of the zwitterionic compound of formula Ij with one additional equivalent of an appropriate base provides the zwitterion, mono salt form of formula Ik. Treatment of the zwitterion, mono salt of formula Ik with an additional one or more equivalents of an appropriate base can provide the zwitterion, bis salt of formula Ii. Likewise, treatment of a compound of formula Il can be converted to the compounds of formulae Ik, Ij, or Ii by treatment with one, two, or three or more equivalents of an appropriate acid, respectively.

[chemical structure]

The compounds of the present invention can be made by conventional methods. A suitable procedure is summarized as depicted below in Scheme 4. The carboxyl-protecting group, R, as shown in a compound of formula V, is intended to include readily removable ester groups which have been conventionally employed to block a carboxyl group during the reaction steps used to prepare the compounds of the present invention and which can be removed by methods which do not result in any appreciable destruction of the remaining portion of the molecule, e.g. by chemical or enzymatic hydrolysis, treatment with chemical reducing agents under mild conditions, irradiation with ultraviolet light or catalytic hydrogenation. Examples of such protecting groups include benzhydryl, p-nitrobenzyl, 2-naphthylmethyl, allyl, benzyl, p-methoxybenzyl, trichloroethyl, silyl such as trimethylsilyl, phenacyl, acetonyl, o-nitrobenzyl, 4-pyridylmethyl and C₁-C₆ alkyl such as methyl, ethyl or tertiary-butyl. Included within such protecting groups are those which are hydrolyzed under physiological conditions such as pivaloyloxymethyl, acetoxymethyl, phthalidyl, indanyl, acetoxyethyl, pivaloyloxyethyl and methoxymethyl. Compounds of the present invention with such physiologically hydrolyzable carboxyl protecting groups are also referred to herein as “prodrugs”. Compounds of the present invention where R is a physiologically removable protecting group are useful directly as antibacterial agents. Compounds where an R protecting group is not physiologically removable are useful intermediates which can be easily converted to the bioactive form by conventional deblocking procedures well-known to those skilled in the art.

Compounds of the present invention wherein a hydroxyl substituent is esterified with a group hydrolyzable under physiological conditions are also included within the scope of the term “prodrug” as used herein. Such hydroxyl-protecting groups may be employed, for example, to increase the solubility of a cephem derivative. Illustrative of suitable ester “prodrugs” of this type are compounds wherein one or more hydroxy substituent groups are converted to sulfate (—OSO₃H) or phosphate (—OPO₃H₂) groups.

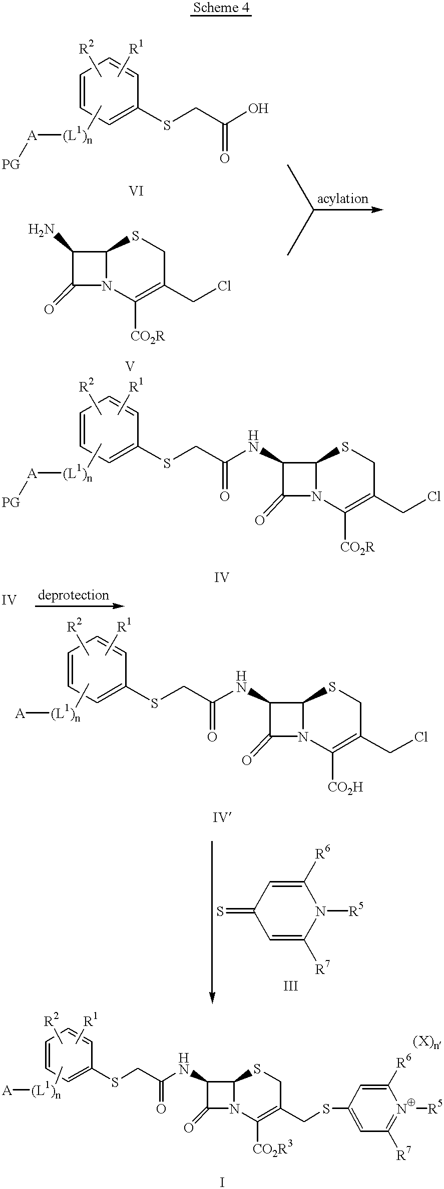

To elaborate on the above process, intermediates of type VI are first prepared, for example, by processes such as those illustrated below in Schemes 5 and 6. Specific embodiments of the methods depicted in Schemes 5 and 6, and reasonable variations upon those methods, are provided hereinafter as described by Schemes I-1 through I-10. The acid intermediate of formula VI is then coupled with a suitable cephem intermediate having a 3-substituent leaving group. For example, the leaving group may be acetoxy or halogen. In the preferred embodiment illustrated in Reaction Scheme 4, the cephem intermediate is the 3-chloromethyl cephem V, but other suitable cephem intermediates with equivalent leaving groups at the 3-position could also be employed. Cephem intermediates such as the intermediate of formula V are well known in the cephalosporin art and are readily accessible. For example, intermediates of formula V wherein R is 4-methoxybenzyl or diphenylmethyl are commercially available as the corresponding hydrochloride salt from suppliers such as Otsuka Chemical Co., Ltd. (Japan). The cephem intermediate V may be acylated with VI or a reactive derivative thereof by conventional acylation procedures well-known in the cephalosporin art to give N-acylated intermediate IV. In addition to using the free arylthioacetic acid, e.g. with a suitable condensing agent such as dicyclohexylcarbodiimide, acylating agent VI may also be employed in the form of equivalent acylating derivatives such as an acid anhydride, mixed anhydride, activated ester, or acid halide. The cephem intermediate preferably has the carboxyl group protected by a conventional carboxyl-protecting group which can be readily removed. Examples of such protecting groups are discussed above and include benzyl, 4-nitrobenzyl, 1,1-dimethylethyl, 4-methoxybenzyl, diphenylmethyl, allyl, and the like. Other examples of suitable protecting groups are disclosed in Protective Groups in Organic Synthesis, 2nd Ed., Theodora W. Greene (John Wiley & Sons, 1991), Chapter 5. In one embodiment, intermediate V may be acylated with acid VI in the presence of dicyclohexylcarbodiimide and in an inert solvent such as tetrahydrofuran or dichloromethane. The reaction temperature is typically between −20° C. and 50° C. Upon completion of the reaction, insoluble material is removed by filtration, the filtrate is concentrated, and the residue is treated with a relatively non-polar solvent such as diethyl ether or ethyl acetate resulting in precipitation of the desired product. In another preferred embodiment, acid VI may be converted to the corresponding acid chloride, for example by treatment with Vilsmeier reagent, [ClCH═N(CH₃)2]⁺Cl⁻ in tetrahydrofuran or ethyl acetate or by treatment with thionyl chloride with or without a solvent such as dichloromethane. The resulting acid chloride can be coupled with the amino cephem of formula V (or its corresponding hydrochloride salt) in the presence of a base such as triethylamine, N-methylmorpholine, or aqueous potassium bicarbonate or sodium bicarbonate to give intermediate IV. Cephem IV is typically isolated after aqueous work-up and evaporation of volatile solvents followed by trituration of the compound with a relatively non-polar solvent such as diethyl ether or ethyl acetate. This intermediate may be used in the next reaction step as the chloromethyl derivative (as shown in Scheme 4), or can be converted to the corresponding bromomethyl or iodomethyl derivative by treatment of the chloromethyl derivative with the appropriate metal halide, such as sodium bromide or sodium iodide, in a solvent such as acetone.

To prepare the quaternary cephem I, intermediate IV is deprotected under acidic conditions, followed by reaction of the resulting intermediate IV′ with a thiopyridone derivative of formula III. For example, when R is diphenylmethyl or 4-methoxybenzyl, cephem acid IV′ is obtained upon treatment of IV with a strong acid, such as trifluoroacetic acid neat or in an inert solvent such as methylene chloride. A reagent such as anisole or triethylsilane may also be employed to scavenge the liberated ester protecting group. The reaction temperature is usually at or below room temperature. The deprotection may also be carried out by treatment with other protic acids such as hydrochloric acid in a solvent such as methanol or dioxane or with formic acid, 98%. The product of formula IV′ is typically isolated by precipitation or crystallization. Reaction of IV′ with a thiopyridone derivative III in a solvent such as dimethylformamide, dimethyl sulfoxide, ethanol, methanol, or other appropriate solvents at a temperature between −20° C. and 100° C. affords target quaternary cephem I. The final product is isolated as described above. It is to be understood that the compound of formula I can exist in various salt forms, some of which have been previously depicted in Schemes 1 through 3.

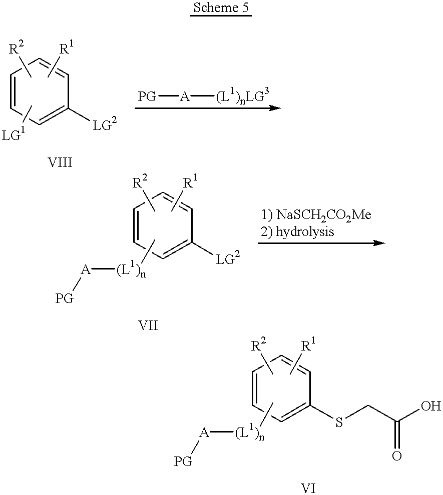

Scheme 5, above, depicts the preparation of the phenylthioacetic acid derivative of formula VI starting from the appropriate benzene derivative of formula VIII. In Scheme 5, LG¹, LG² and LG³ represent appropriate groups which are displaced in the course of the synthesis as shown. For example, LG¹ can represent a halogen, such as iodo, which is then displaced by the moiety represented by PG-A-(L¹)ₙ— using standard organic chemistry methods such as the Heck reaction or the Stille coupling reaction. PG represents an acid protecting group for the group A and in certain preferred embodiments is tertiary-butyl. When PG is tertiary-butyl it may be subsequently deblocked by treatment with a strong acid, such as hydrochloric or trifluoroacetic acid, to provide group A. The group represented by LG³ is displaced from the moiety PG-A-(L¹)ₙ upon its addition to the benzene derivative of formula VIII to provide derivatives of formula VII. For example, LG³ can represent a terminal vinylic hydrogen displaced from an acrylate, such as tertiary-butyl acrylate, during introduction of the group —CH═CHCO₂C(CH₃)₃ using the Heck reaction or LG³ can represent a stannyl group, such as tri-n-butylstannyl, —Sn((CH₂)₃CH₃)₃ or trimethylstannyl, —Sn(CH₃)₃ displaced from a stannane of formula PG-A-(L¹)ₙ—Sn((CH₂)₃CH₃)₃ or PG-A-(L¹)ₙ—Sn(CH₃)₃, respectively, during the Stille coupling reaction. The intermediate of formula VII, wherein LG² can be a halide such as chloro, is then reacted with the sodium salt of methyl mercaptoacetate, followed by hydrolysis, to provide the acid intermediate of formula VI. Further description of the methods used to prepare intermediates of general formula VI is provided hereinafter in Schemes I-1 through I-10.

Scheme 6, below, depicts another method for the preparation of phenylthioacetic acid intermediates of general formula VI starting from an appropriate thiophenol derivative of formula X. The thiophenol derivative of formula X is first reacted with methyl bromoacetate in the presence of a suitable base, such as triethylamine or 4-methylmorpholine, to provide the intermediate of formula IX. The group PG-A-(L¹)ₙ is then introduced by the methods as previously described in Scheme 5, followed by hydrolysis to provide the intermediate of formula VI. Other representative intermediate VI groups may be prepared as described in the preparation of intermediates section below.

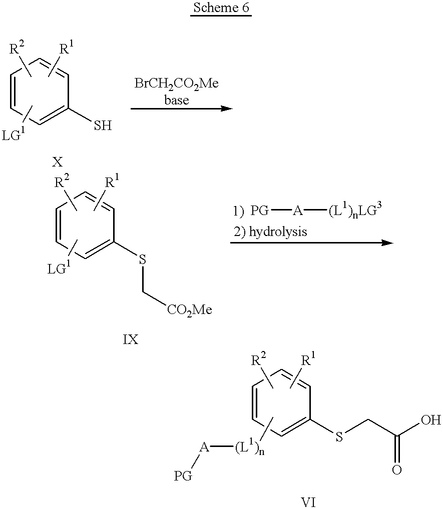

Thiopyridones of formula III are typically prepared according to a method analogous to that described in T. Takahashi et al., European Patent Application No. 209751 and in I. E. El-Kholy et al., J. Heterocyclic Chem. 1974, 11, 487 and as described by Bronson, J. J. et al. in U.S. Pat. No. 5,668,290 (1997) which is hereby incorporated by reference in its entirety. As depicted below in Scheme 7 this procedure entails reaction of an appropriate 4-thiopyrone (U.S. Pat. No. 5,668,290 and European Patent No. 209751) with an appropriate primary amine, R⁵—NH₂, in a solvent such as aqueous methanol, ethanol, or N,N-dimethylformamide at a temperature ranging between 0° C. and 78° C. The primary amine may be in the form of a zwitterion in examples where there is a free acid group present in the molecule. In these cases, a base such as sodium hydroxide, sodium bicarbonate, or pyridine is added to form the free amine in situ. The product may be isolated as its sodium salt by evaporation of volatile solvents, followed by trituration with a solvent such as diethyl ether or ethyl acetate. Alternatively, the reaction mixture may be acidified and extracted with an organic solvent to afford the product as the free carboxylic acid. If the carboxylate group is protected as an ester, the amine may be free or present as an acid salt. In the latter case, a base such as sodium hydroxide, sodium bicarbonate, or pyridine is added to form the free amine in situ. The product is typically isolated by precipitation or by reversed phase column chromatography following removal of volatile solvents. The amine of formula R⁵NH₂ may also contain a quaternary nitrogen moiety such as a quaternary morpholine or imidazole and a corresponding counter anion. Reaction of such an amine with 4-mercaptopyrone results in the corresponding 4-thiopyridone of formula III which contains the quaternary nitrogen moiety and its counter anion.

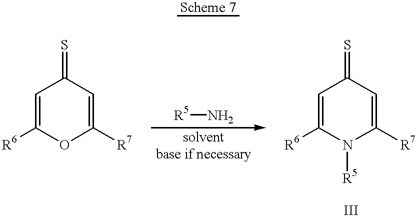

It will be understood that where the substituent groups used in the above reactions contain certain reaction-sensitive functional groups such as amino or carboxylate groups which might result in undesirable side-reactions, such groups may be protected by conventional protecting groups known to those skilled in the art. For example, thiopyridone intermediates of formula III may have an amine functional group protected as the t-butyloxycarbamate. Suitable protecting groups and methods for their removal are illustrated, for example, in Protective Groups in Organic Synthesis, 2^{nd} Ed., Theodora W. Greene (John Wiley & Sons, 1991). It is intended that such “protected” intermediates and end-products are included within the scope of the present disclosure and claims.

The desired end-product of formula I may be recovered in various salt forms as previously shown in Schemes I through 3. For example, the compound of formula I may be isolated as the zwitterion or in the form of a pharmaceutically acceptable acid addition salt, e.g. by addition of an appropriate acid such as HCl, HI or methanesulfonic acid to the zwitterion, or in certain cases as base addition salts. Compounds of formula I where R³ is hydrogen or an anionic charge, or a pharmaceutically acceptable salt thereof, may be converted by conventional procedures to a corresponding compound where R³ is a physiologically hydrolyzable ester group.

It will be appreciated that certain products within the scope of Formula I may have a C-3 substituent group which can result in formation of optical isomers. It is intended that the present invention include within its scope all such optical isomers as well as epimeric mixtures thereof, i.e. R- or S- or racemic forms.

The novel cephalosporin derivatives of general formula I wherein R³ is hydrogen, an anionic charge or a physiologically hydrolyzable carboxyl-protecting group, or prodrugs thereof, are potent antibiotics active against many gram-positive bacteria. While they may be used, for example, as animal feed additives for promotion of growth, as preservatives for food, as bactericides in industrial applications, for example in water-based paint and in the white water of paper mills to inhibit the growth of harmful bacteria, and as disinfectants for destroying or inhibiting the growth of harmful bacteria on medical and dental equipment, they are especially useful in the treatment of infectious disease in humans and other animals caused by the gram-positive bacteria sensitive to the new derivatives. Because of their excellent activity against MRSA organisms, they are particularly useful in the treatment of infections resulting from such bacteria.

The pharmaceutically active compounds of this invention may be used alone or formulated as pharmaceutical compositions comprising, in addition to the active cephem ingredient, a pharmaceutically acceptable carrier or diluent. The compounds may be administered by a variety of means, for example, orally, topically or parenterally (intravenous or intramuscular injection). The pharmaceutical compositions may be in solid form such as capsules, tablets, powders, etc. or in liquid form such as solutions, suspensions or emulsions. Compositions for injection, the preferred route of delivery, may be prepared in unit dose form in ampules or in multidose containers and may contain additives such as suspending, stabilizing and dispersing agents. The compositions may be in ready-to-use form or in powder form for reconstitution at the time of delivery with a suitable vehicle such as sterile water.

The dosage to be administered depends, to a large extent, on the particular compound being used, the particular composition formulated, the route of administration, the nature and condition of the host and the particular situs and organism being treated Selection of the particular preferred dosage and route of application, then, is left to the discretion of the physician or veterinarian. In general, however, the compounds may be administered parenterally or orally to mammalian hosts in an amount of from about 50 mg/day to about 20 g/day. Administration may be once a day, although it is generally carried out in divided doses, e.g., two to four times a day, analogous to dosing with a cephalosporin such as cefotaxime.

### IN VITRO ACTIVITY

Samples of the compounds prepared below in Examples 1-22 after solution in water and dilution with Nutrient Broth were found to exhibit the following Minimum Inhibitory Concentrations (MIC) values versus the indicated microorganisms as determined by tube dilution. The MICs were determined using a broth micro dilution assay in accordance with that recommended by the National Committee for Clinical Laboratory Standards (NCCLS). Mueller-Hinton medium was used except for Streptococci which was tested in Todd Hewitt broth. The final bacterial inoculate contained approximately 5×10⁵ cfu/ml and the plates were incubated at 35° C. for 18 hours in ambient air (Streptococci in 5% CO₂). The MIC was defined as the lowest drug concentration that prevented visible growth.

| Microorganism | MIC value in ug/ml |
|---|---|
| S. aureus methicillin resistant A27223 | ≦8 |
| S. pneumoniae A9585 | ≦2 |
| S. pyogenes A9604 | ≦2 |
| E. faecalis A20688 | ≦8 |
| S. aureus A9537, penicillinase negative | ≦1 |
| S. aureus A15090, penicillinase positive | ≦1 |
| S. epidermidis A24548 | ≦1 |
| S. epidermidis A25783 methicillin resistant | ≦2 |
| S. hemolyticus A21638 | ≦8 |

### IN VIVO ACTIVITY

The in vivo therapeutic efficacy of the compounds prepared in Examples 1-22 below after intramuscular injection to mice experimentally infected with the representative MRSA strain A27223 was also measured.

The determination of the effectiveness of antimicrobial agents in Staphylococcus aureus systemic infection in mice

Organisms: The test organism, MRSA strain A27223 used to generate systemic infection in mice, is grown on two large Brain Heart Infusion Agar plates. On each plate, 0.5 ml of frozen stock culture is plated out. Plates are then incubated for 18 hours at 30° C. The next day each plate is washed with 20 ml of Brain Heart Infusion Broth and then pooled together. A microscopic direct count of microorganism is done using a 1:1000 dilution of plate wash. After a direct count is obtained, the number of organisms per milliliter is calculated. The count is adjusted to the desired amount of inoculum by diluting in 4% hog mucin. The desired challenge (amount of organisms given to mice) is 2.4×10⁸ cfu/0.5 ml/mouse for MRSA strain A27223. The mice are infected intraperitoneally with 0.5 ml of challenge. Ten non-treated infected mice are used as controls.

Mice: Mice used are male ICR mice. The average weight of the animals is from 20 to 26 grams.

Drug preparation and treatment: Compounds are tested at 4 dose levels, (25, 6.25, 1.56, and 0.39 mg/kg) and prepared in 5% cremophor, unless otherwise specified. Vancomycin is used as the control compound, and is dosed at 6.25, 1.56, 0.39, and 0.098 mg/kg. It is prepared in 0.1 M phosphate buffer. There are five infected mice per dose level, and they are treated with 0.2 ml of the test compound, preferably by intramuscular injection. Treatment begins 15 minutes and 2 hours post-infection.

Test duration: A PD₅₀ (the dose of drug given which protects 50% of mice from mortality) experiment runs for 5 days. During this time, mortality of mice are checked every day and deaths are recorded. The cumulative mortality at each dose level is used to calculate a PD₅₀ value for each compound. Surviving mice are sacrificed at the end of day 5 by CO₂ inhalation.

Calculation: Actual calculation of PD₅₀ is performed with a computer program using the Spearman-Karber procedure.

Results: The in vivo efficacy, expressed as the PD₅₀ value, ranged from about 0.8 to 22.0 mg/kg (for certain compounds, more than one test was carried out; the indicated range is for at least one test result when multiple tests were done).

### ILLUSTRATIVE EXAMPLES

### Preparation of Intermediates

The following intermediates are useful for the preparation of compounds of Formula I. The intermediates provided are illustrative of intermediates useful for the preparation of compounds of Formula I and are not intended as a limitation on the scope of intermediates useful for the preparation of compounds of Formula I. Reasonable variations of the methods used to prepare the intermediates are intended to be within the scope of this invention.

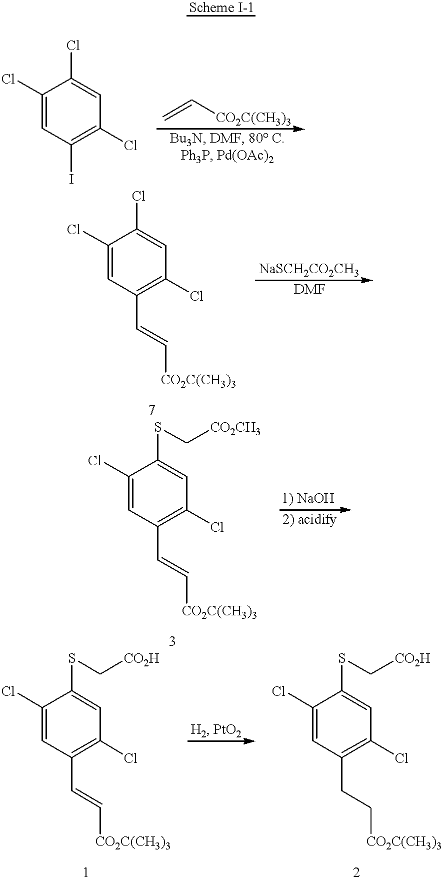

A. Synthesis of Olefin 7

2,4,5-Trichloroiodobenzene (25 g., 81.3 mmol) is dissolved in 80 mL DMF. Tert-Butyl acrylate (48 mL, 328 mmol), tributylamine (58 mL, 243 mmol), triphenylphosphine (4.08 g., 15.5 mmol), and palladium acetate (3.23 g., 14.4 mmol) are added, and the mixture heated to 80° C. for three hours. The solvents are evaporated and the residue is partitioned with EtOAc and water. The aqueous phase is extracted with EtOAc, and the combined organic phase is washed with brine, dried (MgSO₄), and evaporated. The dark red-brown oil is chromatographed on silica in a fritted Buchner funnel using vacuum filtration, with 30% CH₂Cl₂/hexane followed by 50% CH₂Cl₂/hexane as eluant. Acrylate 7 is obtained (18.7 g., 60.8 mmol; 75% yield) as a mauve solid.

¹H-NMR (300 MHz, CDCl₃): δ 7.82 (d, 1H, J=16 Hz, ArCH═C), 7.67 (s, 1H, ArH), 7.51 (s, 1H, ArH), 6.34 (d, 1H, J=16 Hz, C═CHCO₂t-Bu), 1.51 (s, 9H, C(CH₃)₃).

B. Synthesis of Diester 3

Acrylate 7 (21.23 g., 69 mmol) is dissolved in 131 mL DMF. The sodium salt of methyl mercaptoacetate (17.7 g., crude: see note below) is added and the mixture stirred at room temperature for one hour. The mixture is partitioned with EtOAc and water. The aqueous phase is extracted with EtOAc, and the combined organic phase is washed with brine, dried (MgSO₄), and evaporated. Chromatography on silica in a fritted Buchner funnel (vacuum filtration) using 50% CH₂Cl₂/hexane followed by 90% CH₂Cl₂/hexane as eluant affords diester 3 (19.6 g., 52.0 mmol; 75% yield).

¹H-NMR (300 MHz, CDCl₃): δ 7.86 (d, 1H, J=16 Hz, ArCH═C), 7.60 (s, 1H, ArH), 7.36 (s, 1H, ArH), 6.35 (d, 1H, J=16 Hz, C═CHCO₂t-Bu), 3.77 (s, 3H, OCH₃), 3.72 (s, 2H, SCH₂), 1.51 (s, 9H, C(CH₃)₃).

Note: The sodium salt of methyl mercaptoacetate is best made fresh before use. Approximately 30 mL of methyl mercaptoacetate is dissolved in ˜250 mL THF. One equivalent of 5 N NaOH is added slowly in pipetfulls, and the mixture allowed to stir for 5 minutes. The solvents are removed in vacuo (including water) and the sticky solid is co-evaporated with diethyl ether (˜200 mL) and then dry THF (2×200 mL). The solid is pumped dry for several hours under high vacuum until the flask is no longer cool due to evaporation. The freely mobile white solid is used as obtained. Excess of this reagent (1.5 to 2 equivalents) is generally used.

C. Synthesis of Acid 1

Diester 3 (4.40 g, 0.012 mol) is dissolved in 30 mL of THF. To this solution is added 13 mL of 1N NaOH, and the mixture is allowed to stir at room temperature for 1.5 hours. At this time ¹H-NMR analysis of an aliquot indicates that the reaction is complete. The THF is removed under vacuum, and the concentrate is diluted with water and is extracted with EtOAc. The aqueous layer is then acidified with 1 N HCl to pH 4, and then extracted with CH₂Cl₂. The organic phase is washed with brine, dried (MgSO₄), and then evaporated. Acid 1 is obtained as a tan solid (3.80 g, 0.011 mol; 92% yield).

¹H-NMR (300 MHz, CDCl₃): δ 7.82 (d, 1H, J=16 Hz, ArCH═C), 7.56 (s, 1H, ArH), 7.33 (s, 1H, ArH), 6.29 (d, 1H, J=16 Hz, C═CHCO₂t-Bu), 3.72 (s, 2H, SCH₂), 1.51 (s, 9H, C(CH₃)₃).

D. Synthesis of Acid 2

Acid 1 (2.00 g., 0.006 mol) is dissolved in 50 mL EtOH, and PtO₂ (1.00 g., 0.004 mol) is added. The mixture is hydrogenated at 20 psi for 18 hours. ¹H-NMR analysis indicates that the reaction has only proceeded to 30% conversion. More PtO₂ (0.400 g., 0.002 mol) is added, and hydrogenation at 20 psi is continued another 6 hours. At this time the reaction is only 50% complete. The solids are filtered, and the filtrate is treated with fresh PtO₂ (1.00 g., 0.004 mol) and hydrogenated at 20 psi for another 20 hours. The reaction still shows some starting olefin. At this point the mixture is filtered, and the filtrate is concentrated. The residue is chromatographed on silica using CH₂Cl₂ as eluant, followed by a gradient elution with methanol/CH₂Cl₂ (up to 15% MeOH). Acid 2 is obtained (0.750 g., 0.002 mol; 33% yield) as a white solid.

¹H-NMR (300 MHz, CDCl₃): δ 7.40 (s, 1H, ArH), 7.30 (s, 1H, ArH), 3.72 (s, 2H, SCH₂), 2.96 (t, 2H, J=8 Hz, ArCH₂), 2.54 (t, 2H, J=8 Hz, CH₂CO₂R), 1.43 (s, 9H, C(CH₃)₃).

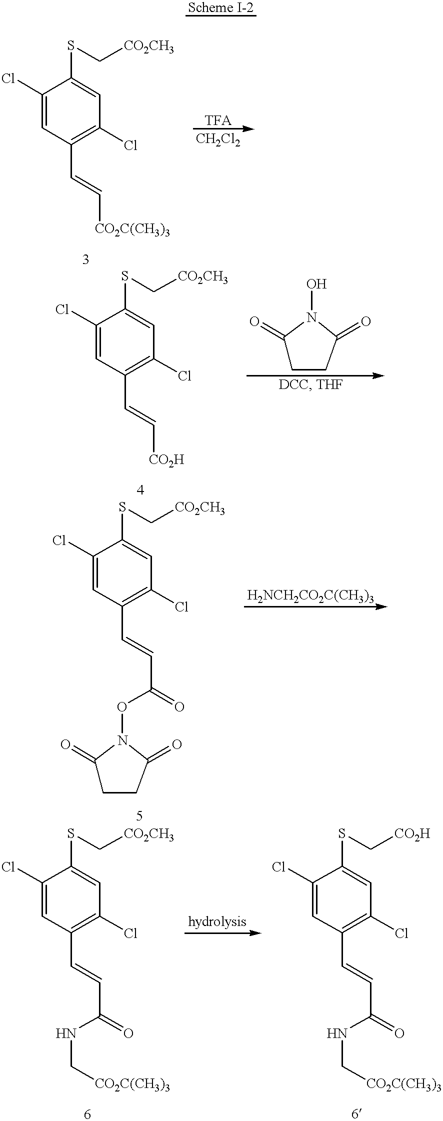

A. Synthesis of Acid 4

Diester 3 (7.00 g., 0.019 mol) is suspended in 40 mL CH₂Cl₂. Anisole (1 mL) is added, followed by 15 mL of trifluoroacetic acid. The mixture is stirred for 1 hour at room temperature. The solvents are concentrated to ˜15 mL, and excess diethyl ether is added to precipitate a white solid. The solid is collected, washed with diethyl ether, and dried under vacuum to yield acid 4 (4.85 g., 0.015 mol; 79% yield).

¹H-NMR (300 MHz, DMSO-d₆): δ 8.08 (s, 1H, ArH), 7.71 (d, 1H, J=16 Hz, ArCH═C), 7.43 (s, 1H, ArH), 6.69 (d, 1H, J=16 Hz, C═CHCO₂), 4.19 (s, 2H, SCH₂), 3.66 (s, 3H, OCH₃).

B. Synthesis of Hydroxysuccinimide Ester 5

Acid 4 (8.75 g., 0.027 mol) is suspended in 55 mL THF under an atmosphere of nitrogen. Dicyclohexylcarbodiimide (1M in CH₂Cl₂, 28.7 mL, 0.029 mol) is added, followed by N-hydroxysuccinimide (3.14 g., 0.027 mol). The reaction is allowed to stir for 3 hours at room temperature. The mixture is diluted with ˜30 mL acetone, filtered to remove dicyclohexylurea, and concentrated to ˜25 mL. A solid forms which is filtered off, and the filtrate is evaporated to dryness. Crude 5 is obtained as a white solid (12.3 g) which is of sufficient purity for use in subsequent reactions.

¹H-NMR (300 MHz, DMSO-d₆): δ 8.27 (s, 1H, ArH), 8.01 (d, 1H, J=16 Hz, ArCH═C), 7.50 (s, 1H, ArH), 7.17 (d, 1H, J=16 Hz, C═CHCO₂), 4.23 (s, 2H, SCH₂), 3.68 (s, 3H, OCH₃), 2.84 (m, 4H, CH₂CH₂).

C. Synthesis of Amide 6

tertiary-Butylglycine ester, hydrochloride (5.19 g., 0.031 mol) is suspended under a nitrogen atmosphere in 60 mL dry DMF. 4-methylmorpholine (3.90 mL, 0.036 mol) is added, and then the mixture is cooled to 0° C. Crude hydroxysuccinimide ester 5 (12.3 g.) is added, and the mixture allowed to stir for 10 minutes at 0° C. The cooling bath is removed and the reaction is stirred for 1 hour. The mixture is concentrated, and the residue dissolved in EtOAc and placed in a separatory funnel. The solution is washed with 0.4 N aqueous HCl, 0.1 N aqueous NaHCO₃, water and then brine. The organic phase is dried (MgSO₄) and evaporated to afford the amide 6 as a light yellow solid (10.4 g., 0.024 mol; 89% yield from acid 4).

¹H-NMR (300 MHz, DMSO-d₆): δ 8.42 (t, 1H, J=8 Hz, NH), 7.81 (s, 1H, ArH), 7.59 (d, 1H, J=16 Hz, ArCH═C), 7.47 (s, 1H, ArH), 6.80 (d, 1H, J=16 Hz, C═CHCONH), 4.20 (s, 2H, SCH₂), 3.85 (d, 2H, J=8 Hz, NCH₂CO₂), 3.67 (s, 3H, OCH₃), 1.41 (s, 9H, C(CH₃)₃).

D. Synthesis of Acid 6′

Diester 6 (0.600 g., 1.40 mmol) is dissolved in 5 mL THF. Aqueous 1 N NaOH (1.40 mL, 1.40 mmol) is added and the mixture is allowed to stir at room temperature for 1 hour. The THF is evaporated, and the residue taken up in 20 mL water. The solution is acidified to pH=3 with 1N HCl, and the mixture is partitioned with EtOAc and water. The organic phase is washed with brine, dried (MgSO₄), and evaporated. Pure acid 6′ is obtained (0.470 g., 1.12 mmol; 80% yield).

¹H-NMR (300 MHz, DMSO): δ 8.45 (t, 1H, J=7 Hz, NH), 7.83 (s, 1H, ArH), 7.59 (d, 1H, J=17 Hz, C═CH), 7.43 (s, 1H, ArH), 6.80 (d, 1H, J=17 Hz, C═CH), 4.08 (s, 2H, SCH₂), 3.85 (d, 2H, J=7 Hz, NCH₂), 1.43 (s, 9H, C(CH₃)₃).

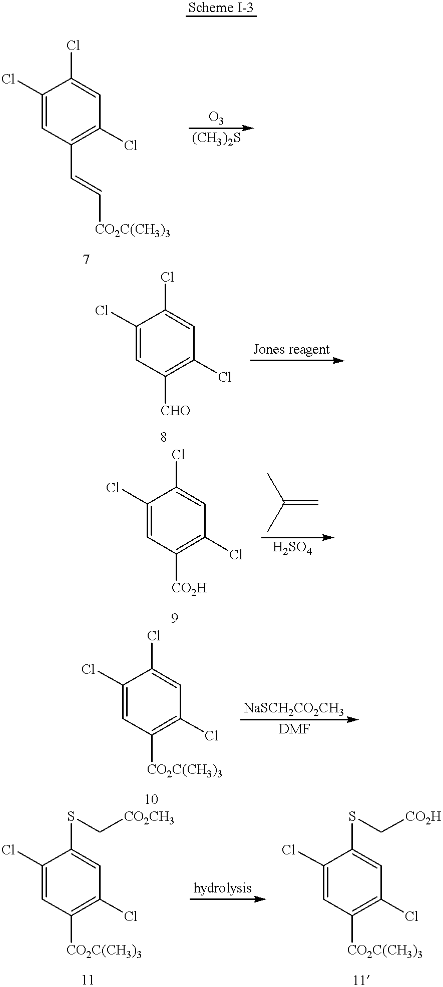

A. Synthesis of 2,4,5-Trichlorobenzaldehyde 8

Ester 7 (1.03 g., 3.35 mmol) is dissolved in 200 mL CH₂Cl₂ and 100 mL methanol. The mixture is cooled to −78° C., and ozone is bubbled through the solution until it turns blue. The mixture is purged with oxygen, and then 1.3 mL of methyl sulfide is added. The mixture is allowed to warm to room temperature, and the solvents are evaporated. The crude residue is partitioned between diethyl ether and water. The diethyl ether layer is washed with water and brine, and then dried (MgSO₄). Concentration of the diethyl ether layer, followed by chromatography of the resulting residue on silica using 25% CH₂Cl₂/hexane as eluant, affords 8 (0.55 g., 2.63 mmol; 79% yield) as a white solid.

¹H-NMR (300 MHz, CDCl₃): δ 10.34 (s, 1H, CHO), 7.98 (s, 1H, ArH), 7.58 (s, 1H, ArH).

B. Synthesis of 2,4,5-Trichlorobenzoic Acid 9

2,4,5-Trichlorobenzaldehyde (0.275 g., 1.31 mmol) is dissolved in 8 mL acetone. A slight excess of Jones Reagent is added, and the mixture stirred at room temperature for 1 hour. Methanol (˜6 mL) is added, and after 5 minutes the mixture is partitioned between methylene chloride and water. The aqueous phase is extracted with chloroform (2×), and the combined organic extracts are washed with water, then brine. The organic phase is dried (MgSO₄), and concentrated to afford pure 2,4,5-trichlorobenzoic acid 9 (0.285 g., 1.26 mmol; 96% yield) as a white solid.

¹H-NMR (300 MHz, CDCl₃): δ 8.01 (s, 1H, ArH), 7.61 (s, 1H, ArH).

C. Synthesis of 2,4,5-Trichlorobenzoic Acid, Tertiary-Butyl Ester 10

Acid 9 (2.93 g., 13.0 mmol) placed in a Parr hydrogenation bottle (under an atmosphere of nitrogen) is dissolved in 55 mL of dioxane. The bottle is cooled to −78° C., and 5 mL conc. Sulfuric acid is added cautiously, followed by ˜50 mL liquid isobutylene (cooled to −78° C.). The bottle is sealed and agitated on a Parr shaker for approximately 19 hours. The sealed bottled is vented, and the solution slowly added to a separatory funnel containing half-saturated aqueous NaHCO₃ and diethyl ether. The aqueous layer is extracted with diethyl ether, and the combined organic phase was dried (MgSO₄) and concentrated. Chromatography of the residue on silica using hexane, followed by 25% CH₂Cl₂/hexane, affords ester 10 as a pale yellow oil (2.56 g., 9.10 mmol; 70% yield) which solidifies overnight in the refrigerator to an off-white solid.

¹H-NMR (300 MHz, CDCl₃): δ 7.81 (s, 1H, ArH), 7.53 (s, 1H, ArH), 1.58 (s, 9H, C(CH₃)₃).

D. Synthesis of Diester 11

Using the method described previously for the synthesis of diester 3, ester 10 (2.5 g., 11.3 mmol) is converted to diester 11 (3.20 g., 9.12 mmol; 81% yield). Diester 11 was obtained as a white solid by chromatography on silica gel using 80% CH₂Cl₂/hexane.

¹H-NMR (300 MHz, CDCl₃): δ 7.75 (s, 1H, ArH), 7.30 (s, 1H, ArH), 3.75 (s, 3H, OCH₃), 3.72 (s, 2H, SCH₂), 1.58 (s, 9H, C(CH₃)₃).

E. Synthesis of Acid 11′

Diester 11 (1.60 g., 4.60 mmol) is dissolved in 12 mL THF. Aqueous 1N NaOH (4.6 mL, 4.60 mmol) is added and the mixture is allowed to stir at room temperature for 1.5 hours. The THF is evaporated, and the residue taken up in ˜40 mL water. The aqueous layer is extracted with EtOAc (compound is in organic layer at this point!), and evaporated to yield a white solid. The material is re-dissolved in EtOAc, and acidified by shaking in a separatory funnel with ˜0.5 N HCl. The organic layer is washed with brine, dried (MgSO₄), and evaporated. Pure acid 11′ is obtained (1.12 g., 3.32 mmol; 72% yield).

¹H-NMR (300 MHz, DMSO): δ 7.80 (s, 1H, ArH), 7.42 (s, 1H, ArH), 4.10 (s, 2H, CH₂), 1.52 (s, 9H, C(CH₃)₃).

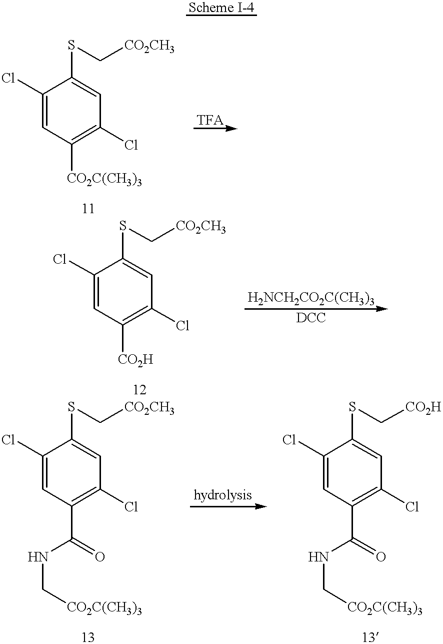

Synthesis of Acid 12

Diester 11 (1.60 g., 4.56 mmol) was dissolved in 5 mL CH₂Cl₂. Trifluoroacetic acid (2 mL) was added to this solution, and the mixture stirred for 4 hours at room temperature. The reaction mixture was concentrated in vacuo to provide 12 (1.24 g., 4.20 mmol; 92% yield) as a tan solid of sufficient purity for use in the next reaction.

¹H-NMR (300 MHz, DMSO-d₆): δ 7.87 (s, 1H, ArH), 7.46 (s, 1H, ArH), 4.23 (s, 2H, SCH₂), 3.68 (s, 3H, OCH₃).

B. Synthesis of Amide 13

Acid 12 (1.24 g., 4.20 mmol) was dissolved in 14 mL dry THF. Dicyclohexylcarbodiimide (0.866 g., 4.20 mmol) was added, followed by tertiary-butyl glycine ester (0.550 g., 4.20 mmol), and the mixture stirred at room temperature for 2 hours. Diethyl ether was added to the flask, and the solids removed by filtration. The filtrate was evaporated to yield 1.96 g. of crude material. Chromatography of the crude material on silica using 80% CH₂Cl₂/hexane, followed by a second chromatography on silica using 20% EtOAc/hexane affords amide 13 (0.844 g., 2.07 mmol; 49% yield) as a white solid.

¹H-NMR (300 MHz, CDCl₃): δ 7.78 (s, 1H, ArH), 7.31 (s, 1H, ArH), 4.12 (d, 2H, J=6 Hz, NCH₂CO₂), 3.77 (s, 3H, OCH₃), 3.72 (s, 2H, SCH₂), 1.48 (s, 9H, C(CH₃)₃).

C. Synthesis of Acid 13′

Diester 13 (0.844 g., 2.07 mmol) is dissolved in 7 mL THF. Aqueous 1N NaOH (2.2 mL, 2.20 mmol) is added and the mixture is allowed to stir at room temperature for 20 minutes. The THF is evaporated, and the residue taken up in 20 mL water. The solution is acidified to a pH of 3 with 1N HCl, and the mixture is partitioned with EtOAc and water. The organic phase is washed with brine, dried (MgSO₄), and evaporated. Pure acid 13′ is obtained (0.770 g., 1.95 mmol; 94% yield).

¹H-NMR (300 MHz, DMSO): δ 8.87 (t, 11H, J=7 Hz, NH), 7.50 (s, 1H, ArH), 7.40 (s, 1H, ArH), 4.07 (s, 2H, SCH₂), 3.86 (d, 2H, J=7 Hz, NCH₂), 1.43 (s, 9H, C(CH₃)₃).

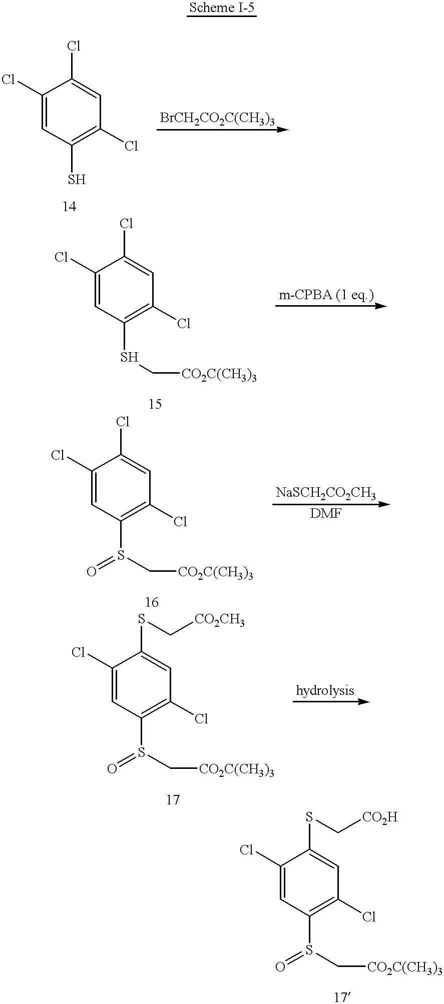

A. Synthesis of Ester 15

2,4,5-Trichlorothiophenol, 14 (35.0 g., 0.166 mol) was dissolved in 500 mL CH₂Cl₂ and cooled to 0° C. Triethylamine (22.0 g., 0.217 mol) was added to the solution, followed by addition of a solution of tertiary-butyl bromoacetate (35.1 g., 0.180 mol) in 100 mL CH₂Cl₂ over a period of 5 minutes. After stirring for 20 minutes at 0° C., the ice-bath was removed, and stirring continued for another hour. The mixture was placed in a separatory funnel and washed with water (2×), 10% aqueous H₃PO₄, and then brine. The organic phase was dried (MgSO₄), and evaporated to afford a white solid, which is washed with hexane. Ester 15 (51 g., 0.156 mol; 94%) as obtained was of suitable purity for subsequent reactions.

¹H-NMR (300 MHz, CDCl₃): δ 7.47 (s, 1H, ArH), 7.45 (s, 1H, ArH), 3.66 (s, 2H, SCH₂), 1.43 (s, 9H, C(CH₃)₃).

B. Synthesis of Sulfoxide 16

Ester 15 (50.0 g., 0.153 mol) is dissolved in 500 mL chloroform and cooled to 0° C. m-Chloroperoxybenzoic acid (50-60% from Aldrich, 48.0 g., 0.140-0.168 mol) is added in small portions over 30 minutes. The ice bath is removed and stirring continued for 2.5 hours at room temperature. The solids were removed by filtration, and the filtrate washed with dilute aqueous NaHSO₃, 5% aqueous Na₂CO₃, saturated aqueous NaHCO₃, and then brine. The organic phase is dried (MgSO₄), and evaporated. The crude material is chromatographed twice on silica gel using CH₂Cl₂ and then 3% methanol/CH₂Cl₂ to afford the sulfoxide 16 (35.0 g., 0.102 mol; 67% yield) as a white solid.

¹H-NMR (300 MHz, CDCl₃): δ 7.95 (s, 1H, ArH), 7.53 (s, 1H, ArH), 4.05 (d, 1H, J=14 Hz, CH₂S(O)), 3.70 (d, 1H, J=14 Hz, CH₂S(O)), 1.43 (s, 9H, C(CH₃)₃).

C. Synthesis of Diester 17

Using the procedure described previously for the synthesis of diester 3, sulfoxide 16 (35.0 g., 105 mmol) is converted to diester 17 (32.5 g., 78.7 mmol; 75% yield). The compound is isolated as a white solid after chromatography on silica using CH₂Cl₂ then 3% methanol/CH₂Cl₂.

¹H-NMR (300 MHz, CDCl₃): δ 7.83 (s, 1H, ArH), 7.28 (s, 1H, ArH), 3.92 (d, 1H, J=14 Hz, CH₂S(O)), 3.77 (s, 3H, OCH₃), 3.71 (s, 2H, SCH₂), 3.57 (d, 1H, J=14 Hz, CH₂S(O)).

D. Synthesis of Acid 17′

Diester 17 (2.00 g., 4.95 mmol) is dissolved in 30 mL THF. Aqueous 1N NaOH (5.7 mL, 5.70 mmol) is added and the mixture is allowed to stir at room temperature for 45 minutes. The THF is evaporated, and the residue is treated with 10% aqueous H₃PO₄ until the solution reaches pH of 4. The solution is extracted with CH₂Cl₂, and the organic layer is washed with water and brine. The organic phase is dried (MgSO₄) and evaporated. Acid 17′ is obtained as a white solid.

¹H NMR (300 MHz, CDCl₃): δ 7.86 (s, 1H, ArH), 7.33 (s, 1H, ArH), 3.94 (d, 1H, J=15 Hz, SOCH), 3.77 (s, 2H, SCH₂), 3.59 (d, 1H, J=15 Hz, SOCH), 1.43 (s, 9H, C(CH₃)₃).

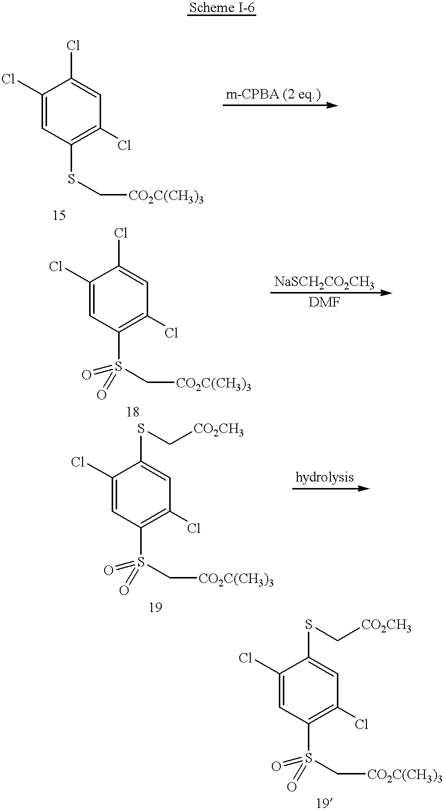

A. Synthesis of Sulfone 18

Ester 15 (7.00 g., 21.4 mmol) was dissolved in 40 mL chloroform and treated with m-chloroperoxybenzoic acid (˜60% from Aldrich, 12.0 g., ˜42 mmol). After stirring for 1 hour at room temperature, the solids are removed by filtration, and the filtrate was washed with dilute aqueous NaHSO₃, 5% aqueous Na₂CO₃, saturated aqueous NaHCO₃, and then brine. The organic phase was dried (MgSO₄), and evaporated. Chromatography of the residue on silica using CH₂Cl₂ then 3% methanol/CH₂Cl₂ as eluent affords the sulfone 18 (7.00 g., 19.5 mmol; 93% yield) as a white solid.

¹H-NMR (300 MHz, CDCl₃): δ 8.17 (s, 1H, ArH), 7.67 (s, 1H, ArH), 4.32 (s, 2H, SCH₂), 1.33 (s, 9H, C(CH₃)₃).

B. Synthesis of Diester 19

Using the procedure described previously for the synthesis of diester 3, sulfone 18 (7.20 g., 20.0 mmol) was converted to diester 19 (8.05 g., 18.8 mmol; 94% yield). The compound was isolated as a white solid after chromatography on silica using CH₂Cl₂ then 3% methanol/CH₂Cl₂ as eluent.

¹H-NMR (300 MHz, CDCl₃): δ 8.05 (s, 1H, ArH), 7.40 (s, 1H, ArH), 4.28 (s, 2H, SO₂CH₂), 3.78 (s, 3H, OCH₃), 3.77 (s, 2H, SCH₂).

C. Synthesis of Acid 19′

Diester 19 (2.00 g., 4.95 mmol) is dissolved in 30 mL THF. Aqueous 1N NaOH (5.7 mL, 5.70 mmol) is added and the mixture is allowed to stir at room temperature for 45 minutes. The THF is evaporated, and the residue is treated with 10% aqueous H₃PO₄ until the solution reaches pH of 4. The solution is extracted with CH₂Cl₂, and the organic layer is washed with water and brine. The organic phase is dried (MgSO₄) and evaporated. Acid 19′ is obtained as a white solid (1.80 g., 4.56 mmol; 92% yield) of suitable purity for coupling to cephem amines.

¹H-NMR (300 MHz, CDCl₃): δ 8.05 (s, 1H, ArH), 7.40 (s, 1H, ArH), 4.30 (s, 2H, SO₂CH₂), 3.80 (s, 2H, SCH₂), 1.31 (s, 9H, C(CH₃)₃).

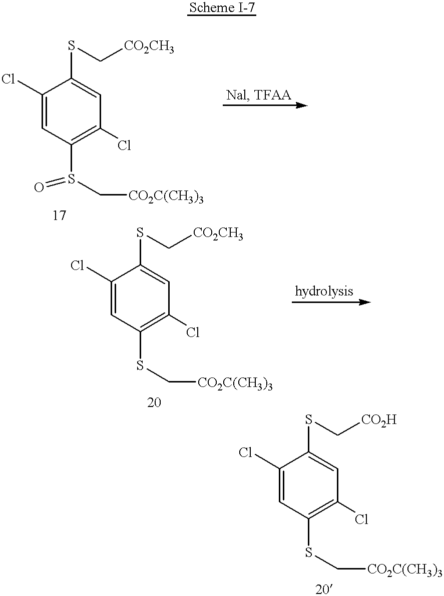

A. Synthesis of Diester 20

Diester 17 (28.0 g., 67.8 mmol) was dissolved in 500 mL acetone. Sodium iodide (48.7 g., 325 mmol) was added, followed by trifluoroacetic anhydride (40.0 g., 191 mmol) over 5 minutes. After stirring at room temperature for 1 hour, the reaction mixture was concentrated in vacuo. CH₂Cl₂ is added to and evaporated from the residue twice. The residue is taken up in CH₂Cl₂ and washed with aqueous NaHSO₃ solution (3×), water, and then brine. The organic phase is dried (MgSO₄), and evaporated. Chromatography of the residue on silica using CH₂Cl₂ affords the diester 20 (23.5 g., 59.2 mmol; 87% yield) as a white solid.

¹H-NMR (300 MHz, CDCl₃): δ 7.38 (s, 1H, ArH), 7.35 (s, 1H, ArH), 3.74 (s, 3H, OCH₃), 3.66 (s, 2H, SCH₂), 3.60 (s, 2H, SCH₂).

B. Synthesis of Acid 20′

Diester 20 (21.0 g., 52.9 mmol) is dissolved in 350 mL THF. Aqueous 1N NaOH (60 mL, 60 mmol) is added and the mixture is allowed to stir at room temperature for 40 minutes. The THF is evaporated, and the residue is treated with 10% aqueous H₃PO₄ until the solution reaches pH of 4. The solution is extracted with CH₂Cl₂, and the organic layer is washed with water and brine. The organic phase is dried (MgSO₄) and evaporated. Acid 20′ is obtained as a white solid (19.3 g., 50.4 mmol; 95% yield) of suitable purity for coupling to cephem amines.

¹H-NMR (300 MHz, CDCl₃): δ 7.40 (s, 1H, ArH), 7.37 (s, 1H, ArH), 3.68 (s, 2H, SCH₂), 3.58 (s, 2H, SCH₂), 1.42 (s, 9H, C(CH₃)₃).

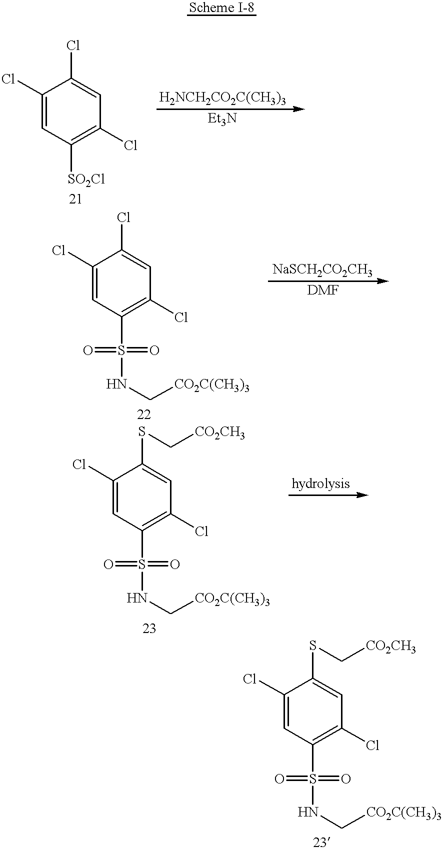

A. Synthesis of Sulfonamide 22

Tertiary-Butyl glycine ester (2.62 g., 20.0 mmol) and triethylamine (2.50 g., 25.0 mmol) were dissolved in 20 mL chloroform under a nitrogen atmosphere. The solution was cooled with an ice-bath, and 2,4,5-trichlorobenzenesulfonyl chloride (5.60 g., 20.0 mmol) dissolved in 30 mL chloroform was added over 5 minutes. The cooling bath was removed and the mixture allowed to stir at room temperature for 1 hour. The solution was washed with 10% aqueous H₃PO₄, water, and then brine. The organic phase was dried (MgSO₄), and evaporated to afford clean sulfonamide 22 (7.00 g., 18.8 mmol; 94% yield) as a white solid.

¹H-NMR (300 MHz, CDCl₃): δ 8.10 (s, 1H, ArH), 7.63 (s, 1H, ArH), 3.72 (s, 2H, NCH₂CO₂), 1.33 (s, 9H, C(CH₃)₃).

B. Synthesis of Diester 23

Using the procedure described previously for the synthesis of diester 3, sulfonamide 22 (3.70 g., 10.0 mmol) is converted to diester 23 (2.37 g., 7.40 mmol; 74% yield). The compound is isolated as a white solid after chromatography on silica using CH₂Cl₂ then 5% methanol/CH₂Cl₂.

¹H-NMR (300 MHz, CDCl₃): δ 7.96 (s, 1H, ArH), 7.36 (s, 1H, ArH), 5.65 (t, 1H, J=5 Hz, NH), 3.85 (s, 3H, OCH₃), 3.78 (s, 2H, SCH₂), 3.71 (d, 2H, J=5 Hz, NCH₂CO₂), 1.34 (s, 9H, C(CH₃)₃).

C. Synthesis of Acid 23′

Diester 23 (1.83 g., 4.24 mmol) is dissolved in 20 mL THF. Aqueous 1N NaOH (15 mL, 15.0 mmol) is added and the mixture is allowed to stir at room temperature for 1 hour. The reaction is concentrated and the aqueous solution is washed with diethyl ether. The aqueous layer is acidified to pH of 4 with 1N HCl, and the solution is extracted with CH₂Cl₂. The organic layer is washed with water and brine. The organic phase is dried (MgSO₄) and evaporated. Acid 23′ is obtained as a white solid (1.40 g., 3.35 mmol; 79% yield) of suitable purity for coupling to cephem amines.

¹H-NMR (300 MHz, CDCl₃): δ 7.98 (s, 1H, ArH), 7.37 (s, 1H, ArH), 5.65 (t, 1H, J=6 Hz, NH), 3.80 (s, 2H, SCH₂), 3.71 (d, 2H, J=6 Hz, NCH₂), 1.33 (s, 9H, C(CH₃)₃).

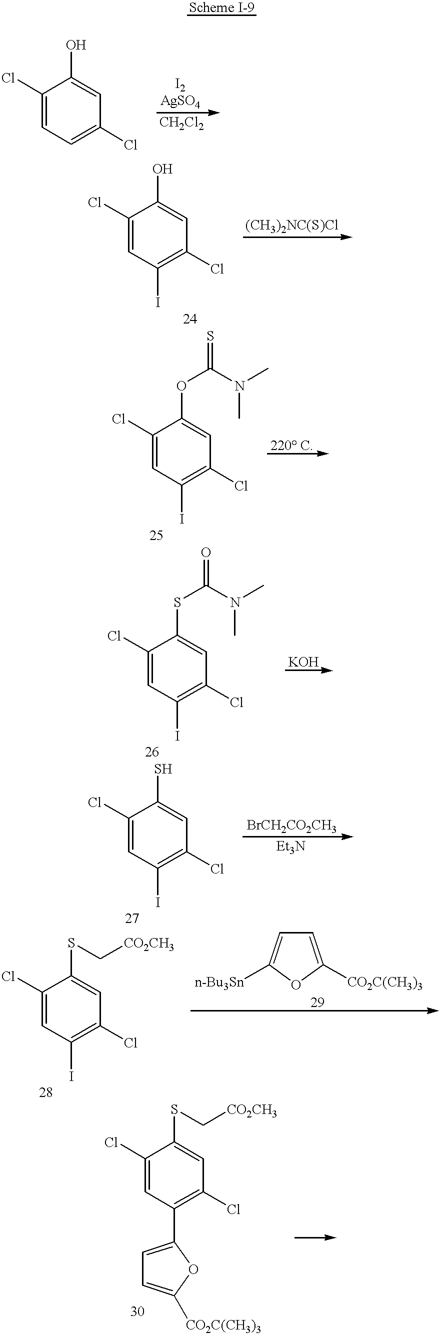

A. Synthesis of 2,5-Dichloro-4-Iodophenol 24

2,5-Dichlorophenol (20.4 g., 0.125 mol) is placed in a 1 L round bottom flask equipped with a large egg-shaped stir bar and is dissolved in 307 mL CH₂Cl₂. With rapid stirring, iodine (46.6 g., 0.183 mol) is added, followed by silver sulfate (42.3 g., 0.136 mol). The purple solution is stirred 1 day, at which point NMR analysis of an aliquot indicates the reaction is complete. The reaction is diluted with CH₂Cl₂ (˜200 mL) and filtered through a fritted Buchner funnel to remove silver salts. The salts are washed with additional CH₂Cl₂ (100 mL). The organic filtrate is transferred to a separatory funnel and is washed first with a solution of sodium thiosulfate (˜40 g. in ˜200 mL water; this removes excess iodine), and then brine. The organic phase is dried (MgSO₄), and evaporated to give 24 (34.59 g., 0.108 mol; 86% yield) as a pale pink/yellow solid.

¹H-NMR (300 MHz, CDCl₃): δ 7.75 (s, 1H, ArH), 7.14 (s, 1H, ArH), 5.62 (br, 1H, OH).

B. Synthesis of Thiocarbamate 25

Iodophenol 24 (34.59 g., 0.108 mol) is placed into a 500 mL round bottom flask equipped with septa, N₂ inlet, and a stir bar. The phenol is then dissolved in 130 mL DMF. DABCO (24.2 g., 0.216 mol) is added followed by dimethylthiocarbamyl chloride (21.6 g., 0.175 mol). The mixture is stirred at room temperature for ˜1 hour, then diluted with EtOAc (˜400 mL) and poured into a separatory funnel containing ˜300 mL of ice-water. The phases are separated, and the aqueous extracted twice with ˜200 mL of EtOAc. The combined organic extracts are washed twice with water (˜100 mL), and then brine. The organic phase is dried (MgSO₄), and evaporated to afford 25 as a dark oil. This material is dissolved in CH₂Cl₂ and dried again (MgSO₄). After evaporation a yellow solid (˜35 g.) is obtained. The compound was of sufficient purity to be used in the next reaction.

¹H-NMR (300 MHz, CDCl₃): δ 7.90 (s, 1H, ArH), 7.24 (s, 1H, ArH), 3.46 (s, 3H, CH₃), 3.37 (s, 3H, CH₃).

C. Synthesis of 26

The crude material obtained above (˜35 g.) was heated neat under N₂ at 220° C. for two hours. After cooling, the material was dissolved in CH₂Cl₂ and filtered through a plug of silica gel. The fractions containing the product are evaporated to afford 30.2 g. of a brown solid. This material was chromatographed on silica (in portions) using a gradient elution starting with 1:1 CH₂Cl₂/hexane (material dissolved in a minimum amount of CH₂Cl₂ for column loading), and then ˜70% CH₂Cl₂hexane. Compound 26 is obtained as a yellow crystalline solid (13.0 g., 36.0 mmol; 33% yield from 2,5-dichlorophenol).

¹H-NMR (300 MHz, CDCl₃): δ 7.95 (s, 1H, ArH), 7.62 (s, 1H, ArH), 3.10 (br s, 3H, CH₃), 3.00 (br s, 3H, CH₃).

D. Synthesis of 2,5-Dichloro-4-Iodothiophenol 27

Carbamate 26 (9.80 g., 0.026 mol) is dissolved in 40 mL EtOH and treated with 30 mL 3N aqueous KOH. The mixture is heated to reflux with stirring under nitrogen for 3 hours. The solution is allowed to cool and is then acidified with 3 N HCl until pH ˜3. The mixture is extracted with CH₂Cl₂ (three times), and the combined organic phase washed with water and then brine. The extracts are dried (MgSO₄) and evaporated. The crude material is chromatographed on silica using 1:1 CH₂Cl₂/hexane. Thiol 27 (6.43 g., 0.021 mol; 81% yield) is obtained as a white solid.

¹H-NMR (300 MHz, CDCl₃): δ 7.83 (s, 1H, ArH), 7.56 (s, 1H, ArH).

E. Synthesis of Ester 28

Thiol 27 (6.43 g., 0.021 mol) is dissolved in 50 mL CH₂Cl₂ and triethylamine (2.52 g., 0.025 mol) is added. Methyl bromoacetate (3.82 g., 0.025 mol) is then added over 5 minutes. The resultant mixture is stirred at room temperature for 1.5 hours, at which time ¹H-NMR analysis indicated the reaction was complete. The mixture was diluted with CH₂Cl₂ (˜200 mL) and was washed with water, 1N HCl, water, and then brine. The organic layer was dried (MgSO₄) and evaporated. The crude material is chromatographed on silica using 70% CH₂Cl₂/hexane. Ester 28 is obtained as a white solid (7.20 g., 0.019 mol; 90% yield).

¹H-NMR (300 MHz, CDCl₃): δ 7.82 (s, 1H, ArH), 7.42 (s, 1H, ArH), 3.75 (s, 3H, OCH₃), 3.68 (s, 2H, SCH₂).

F. Synthesis of Stannane 29

Bis(tributyltin) (29.5 g., 50.9 mmol) is dissolved under a nitrogen atmosphere in 70 mL dry THF. The solution is cooled to −20° C., and butyllithium (1.6 M in hexane, 31.2 mL, 49.9 mmol) is added dropwise over 20 minutes, maintaining the temperature of the bath at −20° C. The solution is then cooled to 50° C., and then copper(I) bromide methylsulfide complex (5.10 g., 24.8 mmol) is added. The mixture is allowed to stir at −40° C. for 15 minutes, and is then cooled to −78° C. 5-Bromofuroic acid tert-butyl ester (4.10 g., 16.6 mmol) dissolved in 15 mL THF is added, and the mixture allowed to stir for 3 hours at −78° C. The reaction mixture is poured into 1 L of diethyl ether and 300 mL half-saturated aqueous ammonium chloride solution. After stirring for 5 minutes the diethyl ether layer is decanted onto another ˜300 mL of half-saturated aqueous ammonium chloride solution. After 5 minutes the biphasic mixture is separated, and the organic phase is washed with brine, dried (MgSO₄) and evaporated. Chromatography on silica using hexane, then 25% CH₂Cl₂/hexane affords stannane 29 (5.05 g., 11.1 mmol; 67% yield) as a clear oil.

¹H-NMR (300 MHz, CDCl₃): δ 7.04 (d, 1H, J=4 Hz, HetArH), 6.56 (d, 1H, J=4 Hz, HetArH), 1.59-1.47 (m, 3 H, SnBu₃), 1.37-1.24 (m, 9 H, SnBu₃), 1.13-1.05 (m, 6 H, SnBu₃), 0.89 (t, 9H, J=6 Hz, SnBu₃).

G. Synthesis of Diester 30

Stannane 29 (1.50 g., 3.28 mmol) is dissolved in 8 mL dry THF. Aryl iodide 28 (0.928 g., 2.46 mmol) is added, followed by bis(triphenylphosphine)-palladium(II) chloride (0.160 g., 0.228 mmol). The solution is heated to reflux for 6 hours. The mixture is diluted with ˜15 mL THF, 4 mL conc. Aqueous KF is added, and the mixture is stirred for 20 minutes. Diethyl ether is added, and the mixture is then filtered to remove insoluble tin solids. The biphasic filtrate is separated, and the aqueous layer is extracted with diethyl ether. The combined organic phases are washed with brine, dried (MgSO₄) and evaporated. During evaporation crystals began to form, and when only ˜5 mL of liquid remains it is decanted. The solids are washed with hexane and then pumped dry. Diester 30 (0.793 g., 1.91 mmol; 78% yield) is obtained as a white solid.

¹H-NMR (300 MHz, CDCl₃): δ 7.99 (br s, 1H, ArH), 7.40 (br s, 1H, ArH), 7.19 (d, 1H, J=2 Hz, HetArH), 7.13 (d, 1H, J=2 Hz, HetArH), 3.75 (s, 3H, OCH₃), 3.71 (s, 2H, SCH₂), 1.60 (s, 9H, C(CH₃)₃).

H. Synthesis of Acid 30′

Diester 30 (0.793 g., 1.91 mmol) is dissolved in 8.65 mL methanol and 10 mL THF. Aqueous 1N NaOH (2.01 mL, 2.01 mmol) is added and the mixture is allowed to stir at room temperature for 20 minutes. The reaction mixture is partitioned with CHCl₃ and aqueous 0.5 N HCl. The aqueous phase is extracted with CHCl₃, and the combined organic phase is washed with brine, dried (MgSO₄), and evaporated. Acid 30′ is obtained (0.771 g., 1.91 mmol; quantitative yield).

¹H-NMR (300 MHz, CDCl₃): δ 8.00 (s, 1H, ArH), 7.40 (s, 1H, ArH), 7.17 (d, 1H, J=4 Hz, furyl H), 7.12 (d, 1H, J=4 Hz, furyl H), 3.76 (s, 2H, CH₂), 1.56 (s, 9H, C(CH₃)₃).

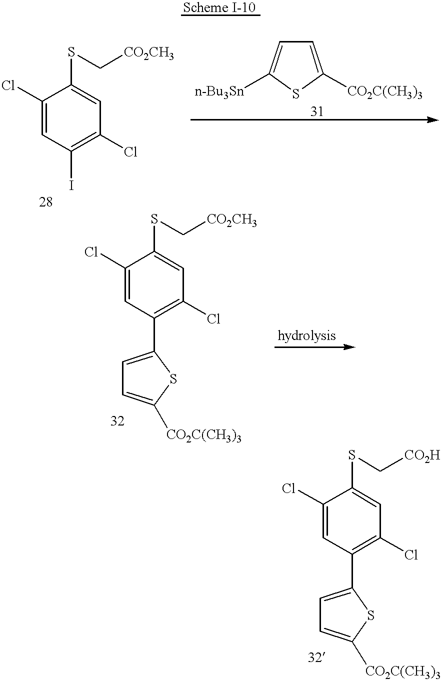

A. Synthesis of Stannane 31

Using the method described above for the synthesis of stannane 29, 5-bromo-2-thiophenecarboxylic acid, tertiary-butyl ester (4.52 g., 17.2 mmol) is converted to stannane 31 (4.33 g., 9.16 mmol; 53% yield). The compound is isolated as a light yellow oil after chromatography on silica using 25% CH₂Cl₂/hexane.

¹H-NMR (300 MHz, CDCl₃): δ 7.80 (d, 1H, J=3 Hz, HetArH), 7.12 (d, 1H, J=3 Hz, HetArH), 1.58 (m, 15H, SnBu₃), 0.92 (t, 9H, J=6 Hz, SnBu₃), 0.85-0.80 (m, 3H, SnBu₃).

B. Synthesis of Diester 32

Using the method described above for the synthesis of diester 30, stannane 31 (1.70 g., 3.60 mmol) and aryl iodide 28 (1.03 g., 2.73 mmol) are converted to diester 32 (0.920 g., 2.13 mmol; 78% yield as a light yellow solid).

¹H-NMR (300 MHz, CDCl₃): δ 7.66 (d, 1H, J=3 Hz, HetArH), 7.55 (s, 1H, ArH), 7.44 (s, 1H, ArH), 7.27 (d, 1H, J=3 Hz, HetArH), 3.78 (s, 3H, OCH₃), 3.73 (s, 2H, SCH₂), 1.58 (s, 9H, C(CH₃)₃).

C. Synthesis of Acid 32′

Diester 32 (1.05 g., 2.43 mmol) was dissolved in 5 mL methanol and 5 mL THF. Aqueous 1N NaOH (2.55 mL, 2.55 mmol) was added. To the nonhomogeneous mixture was added 8 mL of CH₂Cl₂ and the mixture was allowed to stir at room temperature for 20 minutes. The reaction mixture was partitioned with CHCl₃ and aqueous 0.5 N HCl. The aqueous phase was extracted with CHCl₃, and the combined organic phase was washed with brine, dried (MgSO₄), and evaporated. Acid 32′ was obtained (1.05 g., 2.51 mmol; 98% yield) of suitable purity for coupling to cephem amines.

¹H-NMR (300 MHz, CDCl₃): δ 7.68 (d, 1H, J=4 Hz, thiophene H), 7.55 (s, 1H, ArH), 7.45 (s, 1H, ArH), 7.29 (d, I1H, J=4 Hz, thiophene H), 3.77 (s, 2H, CH₂), 1.59 (s, 9H, C(CH₃)₃).

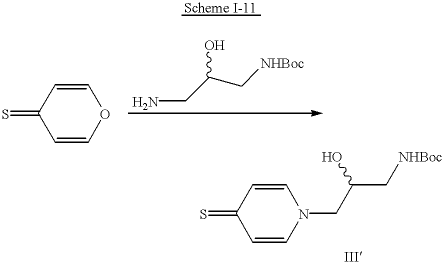

Synthesis of Thiopyridone III′

A mixture of pyran-4-thione (1.10 g., 10.0 mmol) and 1-(N-boc)amino-3-amino-2-propanol (1.90 g., 10.0 mmol) in 25 mL absolute ethanol is stirred for 18 hours at room temperature. The solution is concentrated, and the residue is triturated with a small amount of diethyl ether and filtered to provide thiopyridone III′ (0.850 g., 3.01 mmol; 30%) as a tan solid (more material is present in the filtrate).

¹H-NMR (300 MHz, DMSO): δ 7.54 (d, 2H, J=7 Hz, C═CH), 7.14 (d, 2H, J=7 Hz, C═CH), 6.88 (br t, 1H, J=6 Hz, NH), 5.35 (d, 1H, J=6 Hz, OH), 4.06 (d, 1H, J=13 Hz, NCH), 3.82-3.65 (m, 2H), 2.96 (br dd, 2H, J₁=J₂=6 Hz, NCH₂), 1.39 (s, 9H, C(CH₃)₃).

[chemical structure]

Synthesis of Thiopyridone III″

A. 3-Bromopropylamine hydrobromide (20.5 g., 93.6 mmol) and triphenylmethyl chloride (25.0 g., 103 mmol) were dissolved in 200 mL CH₂Cl₂. A solution of 25 g of triethylamine in 10 mL of CH₂Cl₂ was added dropwise, and the mixture was stirred for 2 hours. The mixture was then washed with water, 10% phosphoric acid, water, and then brine. The organic phase was dried (MgSO₄), and concentrated in vacuo. Upon concentration, a solid formed that was collected by filtration and washed with diethyl ether and hexane to provide 1-triphenylmethyl-3-bromopropylamine (30.0 g., 87.2 mmol; 93% yield).

B. 1-Triphenylmethyl-3-bromopropylamine (6.87 g., 20.0 mmol) and 1,2-dimethylimidazole (2.2 g., 22.9 mmol) were dissolved in 80 mL DMF and heated at 80° C. for 3 hours. The reaction mixture was concentrated in vacuo, and then the residue was diluted with diethyl ether. A precipitate formed that was collected by filtration and washed with diethyl ether to provide 4.5 grams of product. The filtrate was concentrated in vacuo, then treated with a small amount of diethyl ether to afford an additional 2.2 grams of product. The solids are combined to yield 1-(3-triphenylmethylamino)propyl-2,3-dimethylimidazolium bromide (6.70 g., 15.3 mmol; 77%).

C. 1-(3-Triphenylmethylamino)propyl-2,3-dimethylimidazolium bromide (6.50 g., 14.8 mmol) was dissolved in 70 mL methanol and cooled to 0° C. 3N HCl (15 mL) was added, and the cooling bath was removed. The reaction mixture was stirred 30 minutes at ambient temperature, and then the organic solvents were evaporated. The aqueous phase was washed with diethyl ether (2 times) and then with CH₂Cl₂ (3 times), and then concentrated in vacuo to dryness. The 1-(3-amino)propyl-2,3-dimethylimidazolium dichloride obtained (2.30 g., 10.2 mmol; 69%) was used in subsequent reactions without further purification.

D. 1-(3-Amino)propyl-2,3-dimethylimidazolium dichloride (2.30 g., 10.2 mmol) and 2,6-dimethyl-pyran-4-thione (1.6 g., 11.4 mmol) were dissolved in 30 mL absolute ethanol. 2N NaOH (6.3 mL, 12.6 mmol) was added, and the reaction mixture was stirred at room temperature for 18 hours. The reaction mixture was then heated at 60° C. for 6 hours. The mixture was concentrated in vacuo, and acidified with 1N HCl to pH ˜2. The mixture was extracted with CH₂Cl₂ (3 times), and the combined organic extracts were washed with water and then brine. The organic layer was dried (MgSO₄), and concentrated in vacuo. The residue was filtered through a short plug of silica gel by eluting with 5% methanol/CH₂Cl₂ as the solvent. Thiopyridone III″ was obtained as the chloride salt (1.70 g., 5.46 mmol; 54%).

¹H-NMR (300 MHz, DMSO): δ 7.72 (s, 1H, imidazolium), 7.61 (s, 1H, imidazolium), 7.03 (s, 2H, 2×C═CH), 4.23 (t, 2H, J=8 Hz, NCH₂), 4.00-3.92 (m, 2H, NCH₂), 3.75 (s, 3H, NCH₃), 2.69 (s, 3H, CH₃), 2.37 (s, 6H, 2×CH₃), 2.18-2.02 (m, 2H, CH₂).

[chemical structure]

Synthesis of Thiopyridone III″′

1-(3-Amino)propyl-2,3-dimethylimidazolium monochloride (2.26 g., 10.0 mmol) and pyran-4-thione 1.1 g., 9.82 mmol) were dissolved in 20 mL ethanol and stirred 18 hours at room temperature. The solvent was evaporated, and the residue was chromatographed on C-18 silica gel using acetonitrile/water as eluent to afford thiopyridone III″′.

¹H-NMR (300 MHz, DMSO): δ 7.61 (s, 1H, ArH), 7.42 (app s, 2H, 2× imidazolium), 7.59 (d, 2H, J=7 Hz, 2×C═CH), 7.16 (d, 2H, J=7 Hz, 2× C═CH), 4.13 (t, 2H, J=8 Hz, NCH₂), 4.10 (t, 2H, J=8 Hz, NCH₂), 3.72 (s, 3H, NCH₃), 2.55 (s, 3H, CH₃), 2.30-2.15 (m, 2H, CH₂).

[chemical structure]

Synthesis of thiopyridone III″″

5-Formyl-2-furansulfonic acid, sodium salt (1.90 g., 9.59 mmol) and hydrazine hydrate (2 mL) were dissolved in 20 mL absolute ethanol. The mixture was then heated at reflux for 1 hour. The mixture was concentrated in vacuo and the residue was triturated with diethyl ether. Ethanol is added to give the hydrazone shown as a colorless granular solid of suitable purity for further use.

The above hydrazone (3.20 g., 15.2 mmol), pyran-4-thione (1.70 g., 15.2 mmol), and triethylamine (2.19 mL) were dissolved in 40 mL absolute ethanol. The reaction mixture was heated at ˜65° C. for 18 hours then was allowed to cool, and the resulting solids were collected by filtration. The crude solid was dissolved in water and chromatographed on C-18 silica gel using water as eluent. The product containing fractions were frozen and lyophilized to provide thiopyridone III″″ (2.40 g., 7.84 mmol; 52%).

¹H-NMR (300 MHz, DMSO-d₆): δ 8.82 (s, 1H, N═CH), 8.11 (d, 2H, J=7 Hz, 2×C═CH), 7.21 (d, 2H, J=7 Hz, 2×C═CH), 7.16 (d, 1H, J=3 Hz, furyl), 6.66 (d, 1H, J=3 Hz, furyl).

[chemical structure]

Synthesis of Thiopyridone III^{v}

A. To a solution of 4-(3-aminopropyl)morpholine (50.0 g, 0.347 mol) in CH₂Cl₂ (250 mL) at approximately 0° C. was added a solution of Di-tert-butyl dicarbonate (75.5 g, 0.347 mol) in CH₂Cl₂ (250 mL). The reaction mixture was stirred at 0° C. for 1 hour then at room temperature for 14 hours. The reaction mixture was then washed with saturated aqueous NaHCO₃ and water. The organic layer was then dried over Na₂SO₄, filtered and the filtrate concentrated in vacuo to provide 4-(3-(tert-butoxycarbonylamino)propyl)morpholine as a clear oil.

¹H-NMR (300 MHz, DMSO-d₆): δ 6.75 (t, 1H, J=7 Hz), 3.53 (t, 4H, J=5 Hz), 2.91 (q, 2H, J=7 Hz), 2.29 (t, 4H, J=5 Hz), 2.22 (t, 2H, J=7 Hz), 1.35 (s, 9H).

B. To a solution of 4-(3-(tert-butoxycarbonylamino)propyl)morpholine (86 g, 0.352 mol) in acetone (250 mL) was added a solution of methyl iodide (65 g, 0.458 mol) in acetone (102 mL) at room temperature. The reaction mixture was then refluxed for 4 hours, then was allowed to cool to room temperature and was concentrated in vacuo to remove acetone and excess methyl iodide. The residue was triturated with diethyl ether to provide 4-(3-(tert-butoxycarbonylamino) propyl)-4-methyl morpholium iodide as a yellow semi-solid which was used without further purification.

¹H-NMR (300 MHz, DMSO-d₆): δ 6.81 (t, 1H, J=6 Hz), 3.89 (b s, 4H), 3.40-3.52 (m, 6H), 3.15 (s, 3H), 2.96 (q, 2H, J=6 Hz), 1.80 (m, 2H), 1.31 (s, 9H).

C. To a solution of 4-(3-(tert-butoxycarbonylamino) propyl)-4-methyl morpholium iodide (135.8 g) in CH₂Cl₂ at approximately 0° C. was added trifluoroacetic acid (200 mL). The reaction mixture was stirred at 0° C. for 2 hours then at room temperature for 2 hours and then was concentrated in vacuo to remove CH₂Cl₂ and trifluoroacetic acid. 4-(3-ammoniopropyl)-4-methyl morpholinium bis trifluoroacetate was obtained as a viscous oil in quantitative yield.

¹H-NMR (300 MHz, DMSO-d₆): δ 7.75 (b s, 3H), 3.92 (b s, 4H), 3.57 (t, 2H, J=7 Hz), 3.40 (b m, 4H), 3.15 (s, 3H), 2.86 (m, 2H), 1.95 (p, 2H, J=7 Hz).

D. To a solution of 4-(3-ammoniopropyl)-4-methyl morpholinium bis trifluoroacetate (78 mmol) in water (125 mL) and ethanol (200 mL) was added sodium bicarbonate (18 g) in portions. To this solution was added pyran-4-thione (9.6 g, 85 mmol) and ethanol (50 mL). The reaction mixture was stirred for 4 hours at room temperature and then concentrated in vacuo. The residue was triturated with EtOAc followed by diethyl ether to provide the thiopyridone III^{v} as a brown solid.

¹H-NMR (300 MHz, DMSO-d₆): δ 7.66 (d, 2H, J=7 Hz), 7.15 (d, 2H, J=7 Hz), 4.07 (t, 2H, J=5 Hz), 3.90 (b s, 4H), 3.35-3.55 (b m, 6H), 3.15 (s, 3H), 2.20 (m, 2H).

[chemical structure]

Synthesis of Thiopyridone III^{vi}

To a solution of 4-(3-ammoniopropyl)-4-methyl morpholinium bis trifluoroacetate (64.7 mmol) in water (125 mL) and ethanol (250 mL) was added sodium bicarbonate (˜25 g) in portions. To this solution was added 2,6-dimethylpyran-4-thione (9.6 g, 85 mmol). The reaction mixture was stirred for 20 hours at 65° C. and then was allowed to cool to room temperature and was concentrated in vacuo. The residue was triturated with EtOAc, then stirred in EtOAc and the resulting solid was collected by filtration to provide the thiopyridone III^{vi} as a yellow solid.

¹H-NMR (300 MHz, DMSO-d₆): δ 7.07 (s, 2H), 3.98 (t, 2H, J=7 Hz), 3.93 (t, 4H, J=5 Hz), 3.61 (t, 2H, J=7 Hz), 3.44 (t, 4H, J=5 Hz), 3.16 (s, 3H), 2.38 (s, 6H), 2.06 (m, 2H).

Preparation of Compounds of Formula I

The compounds of Formula I may be prepared according to the methods described below. The NMR data and mass spectrometry data for Examples 2-22 is provided in Table 1.

### Example 1

Preparation of 1-[2-hydroxy-3-amino-prop-1-yl]-4-[[(6R)-trans-2-carboxy-8-oxo-7-[(2,5-dichloro-4-(2-carboxyethenyl)phenylthio)acetamido]-5-thia-1-azabicyclo[4.2.0]-oct-2-en-3-yl]methylthio]pyridinium bis trifluoroacetate. The titled compound was prepared according to the methods depicted and described below.

[chemical structure]

A. Synthesis of Cephem IV″

Cephem amine V′ (15.04 g, 0.035 mmol) was suspended under a nitrogen atmosphere in 65 mL of THF. A solution of DCC in CH₂Cl₂ (1.0 M, 36.2 mL, 0.036 mmol) was added, and the mixture was stirred for 5 minutes. Acid 1 (13.15 g, 0.035 mmol) was added and the mixture was stirred for 1.5 hours. Diethyl ether (˜30 mL) was added, and the solids (mostly dicyclohexylurea) were filtered off. The red colored filtrate was concentrated in vacuo to a volume of ˜25-30 mL and diethyl ether and pentane were added to precipitate the cephem product. The resulting solid cephem is collected by filtration, washed with diethyl ether, and dried under vacuum to afford the diester IV″ (14.2 g, 0.019 mol; 54% yield).

¹H-NMR (300 MHz, CDCl₃): δ 7.53 (d, 1H, J=16 Hz), 7.45-7.20 (m, 12H), 6.98 (s, 1H), 6.35 (d, 1H, J=16 Hz), 5.82 (dd, 1H, J=5,8 Hz), 4.97 (d, 1H, J=5 Hz), 4.37 (m, 2H, CH₂Cl), 3.76 (d, 1H, J=16 Hz), 3.55 (d, 1H, J=16 Hz), 3.40 (d, 1H, J=16 Hz), 1.54 (s, 9H).

B. Synthesis of Diacid IV′″

Diester IV″ (0.760 g, 1.00 mmol) was dissolved in 4 mL of CH₂Cl₂ and 0.8 mL anisole. Trifluoroacetic acid (2 mL) was added and the mixture was stirred for 4 hours. The solvents were evaporated and the residue was triturated with CH₂Cl₂/diethyl ether. The solid was collected, washed with EtOAc and then dried under vacuum. Diacid IV′″ was obtained (0.420 g, 0.780 mmol; 78% yield) as a light yellow solid.

¹H-NMR (300 MHz, DMSO): δ 9.30 (d, 1H, J=8 Hz, RC(O)NH), 8.07 (s, 1H, ArH), 7.72 (d, 1H, J=16 Hz, ArCH═C), 7.54 (s, 1H, ArH), 6.68 (d, 1H, J=16 Hz, C═CHCO₂H), 5.71 (dd, 1H, J=5, 8 Hz), 5.13 (d, 1H, J=5 Hz), 4.55 (m, 2H), 3.97 (m, 2H), 3.70 (d, 1H, J=16 Hz), 3.53 (d, 1H, J=16 Hz).

C. Synthesis of Cephem IA′

Diacid IV′″ (0.780 g, 1.45 mmol) was dissolved in 3 mL methanol and 8 mL of CH₂Cl₂. Thiopyridone III′ (0.395 g, 1.45 mmol) was added and the reaction mixture was stirred at room temperature for 4 hours. The reaction mixture was then stored at 4° C. overnight. The reaction mixture was then concentrated in vacuo and the crude product was precipitated with diethyl ether. The crude product was collected by filtration and was then stirred in EtOAc for 30 minutes. The product was then collected by filtration and dried under vacuum to provide the cephem IA′ as a tan solid (0.690 g, 0.850 mmol; 59% yield of a mixture of two diastereomers).

¹H-NMR (300 MHz, DMSO, partial): δ 9.31 (d, 1H, J=8 Hz), 8.67 (d, 2H, J=7 Hz), 8.07 (s, 1H), 7.98 (d, 2H, J=7 Hz), 7.72 (d, 1H, J=16 Hz), 7.54 (s, 1H), 5.70 (dd, 1H, J=5, 8 Hz), 5.14 (d, 1H, J=5 Hz), 4.59-4.45 (m, 2H), 4.40-4.32 (m, 2H), 4.05-3.95 (m, 2H), 1.38 (s, 9H).

D. Synthesis of Cephem IA″

Cephem IA′ (0.605 g, 0.747 mmol) was suspended in 3 mL of CH₂Cl₂ then 1 mL of trifluoroacetic acid was added and the reaction mixture was stirred at room temperature for 2 hours. The reaction mixture was then concentrated in vacuo then the residue was dissolved in CH₂Cl₂ and precipitated with diethyl ether. The solids are collected by filtration, stirred in EtOAc for 30 minutes then again collected by filtration and dried under vacuum in the presence of P₂O₅. Cephem IA″ is obtained (0.410 g, 0.609 mol; 82% yield).

### Example 2

[chemical structure]

Example 2 was prepared according to the same method used to prepare Example 1 with the exception of using acid 2 to acylate the amino cephem V′ instead of acid 1.

### Example 3

Example 3 was prepared as depicted and described below.

[chemical structure]

Diacid IV′″ (41.7 g, 77.5 mmol) and thiopyridone III^{v} (77.5 mmol) were dissolved in 450 mL of DMF and stirred for 1 hour at room temperature then additional diacid IV′″ (2.0 g, 3.7 mmol) was added and the reaction mixture was stirred an additional 15 minutes at room temperature. The reaction mixture was then concentrated in vacuo to remove the DMF. The resulting oily brown solid was then stirred in acetone for 30 minutes and then collected by filtration. The solid was then again stirred in acetone for 30 minutes, collected by filtration, and air dried to provide a brown solid (69 g, crude). The brown solid was suspended in 1.5 L of water and the pH of this aqueous suspension was adjusted to approximately 7-8 by the addition of saturated aqueous NaHCO₃. The aqueous suspension was then purified by reverse phase chromatography on a C-18 column (350 g Waters preparative C-18) using gradient elution (0, 5, 10, 20, 30, 40, 50% acetonitrile/water as mobile phase). Product containing fractions, which eluted in 40-50% acetonitrile/water were combined and concentrated in vacuo in order to remove acetonitrile. After concentration the remaining aqueous fraction was frozen and lyophilized to provide 26.8 g of the product (bis zwitterion form, 4.7 hydrate) as a tan solid. C, H, N Calculated for C₃₂H₃₄N₄O₇S₃Cl₂. 4.7 H₂O: C, 45.58; H, 5.02; N, 6.58; Found: C, 45.84; H, 5.22; N, 6.68.

### Examples 4-7, 11-12

Examples 4 through 7 and 11 and 12 were prepared as depicted in the following reaction scheme and according to the following general procedure.

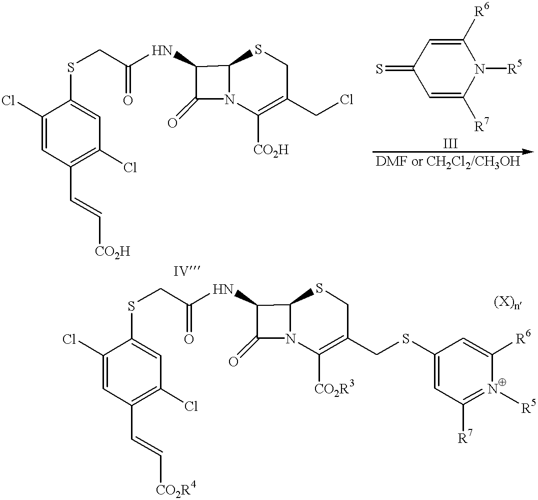

General procedure: Diacid IV′″ (1.0 equivalent) and an appropriate thiopyridone of general formula III (1.0 equivalent) were dissolved in DMF or CH₂Cl₂/CH₃OH and stirred at room temperature for a period of 1 to 10 hours. The reaction mixture was then concentrated in vacuo and the residue was triturated with an appropriate solvent selected from diethyl ether, EtOAc or acetone to provide the product wherein the counterion for the quaternary nitrogen of the pyridinium moiety is chloride (as in Example 7). For examples 4-6 and 11-12 the product thus obtained was then suspended in water and treated with aqueous saturated sodium bicarbonate until the pH of the suspension was 7-8. The product was then purified by reverse phase chromatography on C-18 using gradient elution (typically 0, 5, 10, 20, 30, 40, 50% acetonitrile/water). Product containing fractions were concentrated in vacuo and lyophilized to provide the product as a solid.

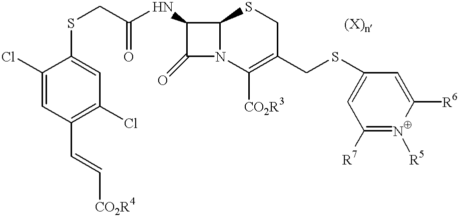

| Example Number | R³ | R⁴ | X | n^{′} | 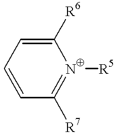 |
|---|---|---|---|---|---|
| 4 | ⊖ | ⊖ | Na^{⊕} | 1 | 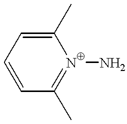 |
| 5 | ⊖ | ⊖ | Na^{⊕} | 1 | 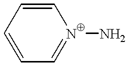 |
| 6 | ⊖ | ⊖ | Na^{⊕} | 1 | 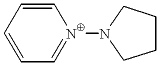 |
| 7 | H | H | Cl^{⊖} | 1 | 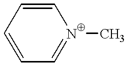 |
| 11 | ⊖ | ⊖ | Na^{⊕} | 2 | [chemical structure] |
| 12 | ⊖ | ⊖ | Na^{⊕} | 2 | [chemical structure] |

### Examples 8-10 and 13-22

Examples 8-10 and 13-22 were prepared as depicted in the following general reaction scheme and according to methods as described previously for Examples 1-7 and 11-12. As shown in step 1, an appropriate acid was coupled to the amino cephem of formula VI using the method previously described for Example 1. The resulting chloromethyl cephem ester was then deprotected and reacted with an appropriate thiopyridone of formula III according to the methods previously described for Examples 1-7 and 11-12.

[chemical structure]

Example 8 was prepared from the corresponding acid which was coupled with amino cephem V′, deprotected, reacted with 2,6-dimethyl-1-(2-hydroxyethyl)-4-thiopyridone, then treated with aqueous sodium bicarbonate and purified as previously described for Examples 4-6 and 11-12.

Examples 9 and 10 were prepared as follows: Acid 20′ was coupled with amino cephem V′, deprotected, then reacted with 1-amino-4-thiopyridone or 1-(2-acetamido)-4-thiopyridone respectively, according to the methods previously described for Examples 1-7 and 11 and 12 to provide the compounds of Examples 9 and 10.

Example 13 was prepared as follows: Acid 11′ was coupled with amino cephem V′, deprotected, then reacted with 2,6-dimethyl-1-[3-(1,2-dimethyl-3-imidazolium)propyl]-4-thiopyridone, chloride salt in DMF according to the methods previously described for Examples 1-7 and 11 and 12 to provide the compound of Example 13.

Example 14 was prepared as follows: Acid 17′ was coupled with amino cephem V′, deprotected, then reacted with 1-(2-hydroxyethyl)-4-thiopyridone according to the methods previously described for Examples 1-7 and 11 and 12 to provide the compound of Example 14.

Example 15 was prepared as follows: Acid 19′ was coupled with amino cephem V′, deprotected, then reacted with 2,6-dimethyl-1-[3-(1,2-dimethyl-3-imidazolium)propyl]-4-thiopyridone, chloride salt in DMF according to the methods previously described for Examples 1-7 and 11 and 12 to provide the compound of Example 15.

Example 16 was prepared as follows: Acid 13′ was coupled with amino cephem V′, deprotected, then reacted with 1-(2-hydroxyethyl)-4-thiopyridone according to the methods previously described for Examples 1-7 and 11 and 12 to provide the compound of Example 16.

Examples 17, 20 and 21 were prepared as follows: Acid 13′ was coupled with amino cephem VI, deprotected, then reacted with 1-(2-hydroxyethyl)-4-thiopyridone, 1-amino-2,6-dimethyl-4-thiopyridone, or 2,6-dimethyl-1-[3 -(4-methyl-4-morpholinium)propyl]-4-thiopyridone, respectively, according to the methods previously described for Examples 1-7 and 11 and 12 to provide the compounds of Examples 17, 20, and 21.

Example 18 was prepared as follows: Acid 30′ was coupled with amino cephem V′, deprotected, then reacted with 1-(2-hydroxyethyl)-4-thiopyridone according to the methods previously described for Examples 1-7 and 11 and 12 to provide the compound of Example 18.

Example 19 was prepared as follows: Acid 32′ was coupled with amino cephem V′, deprotected, then reacted with 2,6-dimethyl-1-[3-(1,2-dimethyl-3-imidazolium)propyl]-4-thiopyridone, chloride salt in DMF according to the methods previously described for Examples 1-7 and 11 and 12 to provide the compound of Example 19.

Example 22 was prepared as follows: Acid 23′ was coupled with amino cephem V′, deprotected, then reacted with 2,6-dimethyl-1-[3-(1,2-dimethyl-3-imidazolium)propyl]-4-thiopyridone, chloride salt in DMF according to the methods previously described for Examples 1-7 and 11 and 12 to provide the compound of Example 22.

Following the procedures described above and using the appropriate starting materials the following compounds of Examples 8-10 and 13-22 were prepared:

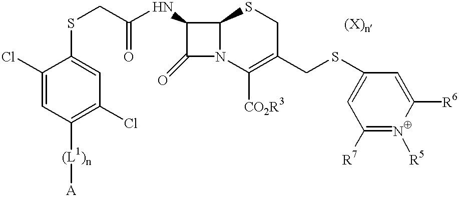

| Example No. | 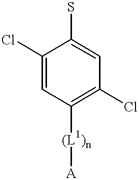 | R³ | X | n^{′} | 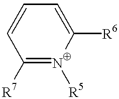 |
|---|---|---|---|---|---|
| 8 | [chemical structure] | ⊖ | Na^{⊕} | 1 | 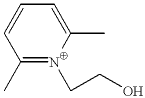 |
| 9 | [chemical structure] | ⊖ | Na^{⊕} | 1 | 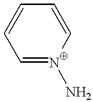 |
| 10 | 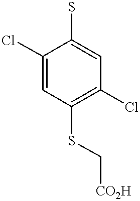 | H | Cl^{⊖} | 1 | 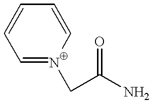 |
| 13 | [chemical structure] | ⊖ | none | 0 | 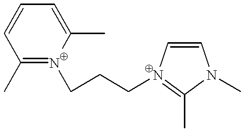 |
| 14 | 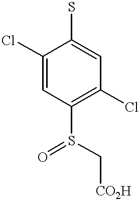 | H | Cl^{⊖} | 1 | 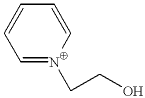 |
| 15 | 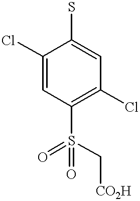 | H | Cl^{⊖} | 2 | 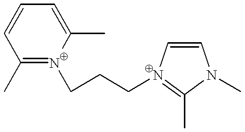 |
| 16 | 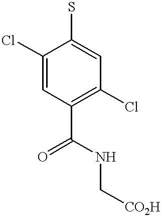 | H | Cl^{⊖} | 1 | 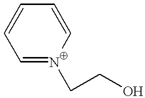 |
| 17 | 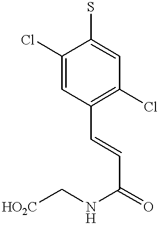 | H | Cl^{⊖} | 1 | 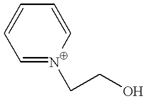 |
| 18 | 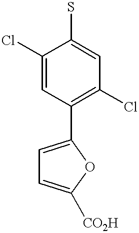 | H | Cl^{⊖} | 1 | 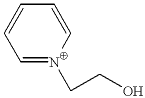 |
| 19 | 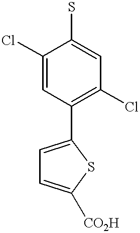 | H | Cl^{⊖} | 2 | 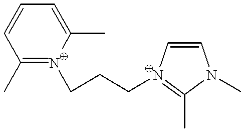 |
| 20 | [chemical structure] | ⊖ | Na^{⊕} | 1 | 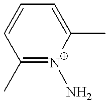 |
| 21 | [chemical structure] | ⊖ | none | 0 | 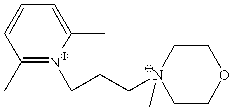 |
| 22 | 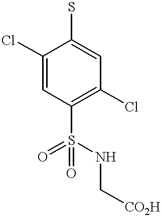 | H | Cl^{⊖} | 2 | 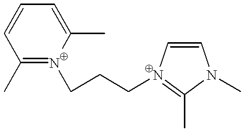 |

TABLE 1
NMR DATA

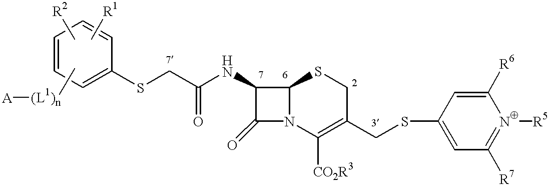

| Compd. of Ex. No. | H-2 | H-6 | H-7 | H-3′ | H-7′ | NH | 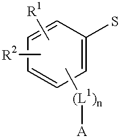 | 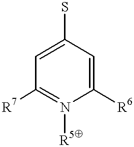 | R⁵ | Isolated Form | MS Data |
|---|---|---|---|---|---|---|---|---|---|---|---|
| 1 | 3.70-3.54 (m) | 5.11 (d, J = | 5.68-5.61 (m) | 4.40-4.39 | 3.97 (br | 9.40-9.35 | 8.12 (s) | 8.77-8.72 (m) | 4.80-4.60 (m) | bis TFA | M⁺ = 685 |
| | | 5) | | (m) | s) | (m) | 7.77 (d, J = 16) | 8.23-8.18 (m) | 4.40-4.24 (m) | salt | |
| | | | | | | | 7.62 (s) | | 4.23-4.10 (m) | | |
| | | | | | | | 6.75 (d, J = 16) | | 3.10-3.00 (m) | | |
| | | | | | | | | | 2.80-2.65 (m) | | |
| 2 | 3.74 (d, J = 18) | 5.13 (d, J = | 5.69 (dd, J = | 4.41-4.37 | 3.95- | 9.24 (d, J = | 7.47 (s) | 8.62 (d, J = 6) | 4.62-4.57 (m) | bis TFA | MH⁺ = 687 |
| | 3.49 (d, J = 18) | 5) | 5,8) | (m) | 3.83 | 8) | 7.45 (s) | 8.05 (d, J = 6) | 4.30-4.23 (m) | salt | |
| | | | | | (m) | | 2.84 (t, J = 7) | | 4.09-3.98 (m) | | |
| | | | | | | | 2.48 (t, J = 7) | | 3.10-2.98 (m) | | |
| | | | | | | | | | 2.80-2.64 (m) | | |
| 3 | 3.92-3.77 (m) | 4.86 (d, J = 5) | 5.39 (d, J = 5) | 4.41 (d, J | 4.09 (br | D₂O exch. | 7.74 (s) | 8.54 (d, J = 7) | 4.50-4.40 (m) | bis | M⁺ = 752 |
| | | | | = 13) | s) | | 7.41 (s) | 7.97 (d, J = 7) | 3.92-3.77 (m) | zwitterion | |
| | | | | 4.29 (d, J | | | 7.39 (d, J = 16) | | 3.49-3.21 (m) | | |
| | | | | = 13) | | | 6.39 (d, J = 16) | | 2.40-2.30 (m) | | |
| 4 | 3.44 (d, J = 17) | 4.90 (d, J = | 5.41 (dd, J = | 4.37 (s) | 3.96 (d, | 9.23 (d, J = | 7.81 (s) | 8.02 (s) | | mono | M⁺ = 654 |
| | 3.26 (d, J = 17) | 5) | 5,8) | | J = 15) | 8) | 7.51 (s) | 2.66 (s) | | sodium | |
| | | | | | 3.88 (d, | | 7.33 (d, J = 16) | | | salt/ | |
| | | | | | J = 15) | | 7.02 (s) | | | zwitterion | |
| | | | | | | | 6.51 (d, J = 16) | | | | |
| 5 | 3.45 (d, J = 17) | 4.94 (d, J = | 5.41 (dd, J = | 4.64 (d, J | 3.92- | 9.23 (d, J = | 7.82 (s) | 8.61 (d, J = 7) | | mono | M⁺ = 626 |
| | 3.29 (d, J = 17) | 5) | 5,8) | = 14) | 3.83 | 8) | 7.50 (s) | 8.23 (d, J = 7) | | sodium | |
| | | | | 4.27 (d, J | (m) | | 7.35 (d, J = 16) | | | salt/ | |
| | | | | = 14) | | | 6.53 (d, J = 16) | | | zwitterion | |
| 6 | 3.46 (d, J = 17) | 4.95 (d, J = | 5.45 (dd, J = | 4.65 (d, J | 3.95 (s) | 9.21 (d, J = | 8.03 (s) | 8.92 (d, J = 7) | 3.42-3.32 (m) | mono | M⁺ = 680 |
| | 3.36 (d, J = 17) | 5) | 5,8) | = 14) | | 8) | 7.68 (d, J = 16) | 8.33 (d, J = 7) | 2.20-1.90 (m) | sodium | |
| | | | | 4.44 (d, J | | | 7.56 (s) | | | salt/ | |
| | | | | = 14) | | | 6.68 (d, J = 16) | | | zwitterion | |
| 7 | 3.76 (d, J = 18) | 5.16 (d, J = | 5.72 (dd, J = | 4.40-4.35 | 4.00 (s) | 9.27 (d, J = | 8.10 (s) | 8.69 (d, J = 6) | 4.18 (s, 3H, | chloride | M⁺ = 628 |
| | 3.56 (d, J = 18) | 5) | 5,8) | (m, 2H) | | 8) | 7.57 (s) | 8.00 (d, J = 6) | CH₃) | salt | |
| | | | | | | | 7.74 (d, J = 16) | | | | |
| | | | | | | | 6.72 (d, J = 16) | | | | |
| 8 | 3.45 (d, J = 16) | 4.85 (d, J = | 5.40 (d, J = 5) | 4.28-4.21 | 4.05 (br | obscured | 7.83 (s) | 7.75 (br s) | 4.45-4.37 (m) | zwitterion | MH⁺ = 754 |
| | 3.24 (d, J = 16) | 5) | | (m) | s) | | 7.55 (d, J = 16) | 2.68 (s) | 3.80-3.75 (m) | mono | |
| | | | | | | | 7.48 (s) | | | sodium | |
| | | | | | | | 6.81 (d, J = 16) | | | salt | |
| | | | | | | | 3.83 (q, J = 8) | | | | |
| | | | | | | | 1.19 (d, J = 8) | | | | |
| 9 | 3.6-3.2 (m) | 4.92 (d, J = | 5.39 (dd, J = | 4.65 (d, J | 3.90- | 9.28 (d, J = | 7.47 (s) | 8.58 (d, J = 7) | | mono | M⁺ = 646 |
| | | 5) | 5,8) | = 13) | 3.70 | 8) | 7.36 (s) | 8.23 (d, J = 7) | | sodium | |
| | | | | 4.25 (d, J | (m) | | | | | salt/ | |
| | | | | = 13) | | | | | | zwitterion | |
| 10 | 3.75 (d, J = 18) | 5.13 (d, J = | 5.68 (dd, J = | 4.40 (d, J | 3.96 (s) | 9.07 (d, J = | 7.65 (s) | 8.63 (d, J = 7) | 7.65 (br s) | chloride | MH⁺ = 689 |
| | 3.53 (d, J = 18) | 5) | 5,8) | = 14) 4.36 | | 8) | 7.39 (s) | 8.01 (d, J = 7) | 5.20 (s) | salt | |
| | | | | (d, J = 14) | | | 3.87 (s) | | | | |
| 11 | 3.6-3.2 (m) | 4.96 (d, J = | 5.42 (d, J = 5) | 4.42-4.19 | 3.95 (d, | D₂O exch. | 7.60 (d, J = 16) | 8.62 (d, J = 7) | 8.88 (s) | Bis sodium | |
| | | 5) | | (m) | J = 14) | | 8.00-7.07 (m) | 8.36 (d, J = 7) | | salt | |
| | | | | | 3.78 (d, | | 6.38 (d, J = 16) | | | | |
| | | | | | J = 14) | | | | | | |
| 12 | 3.76 (d, J = 18) | 5.15 (d, J = | 5.69 (dd, J = | 4.46 (d, J | 3.98 (s) | 9.34 (d, J = | 8.07 (s) | 9.34 (d, J = 6) | 8.04 (d, J = 8) | Bis sodium | MH⁺ = 748 |
| | 3.56 (d, J = 18) | 5) | 5,8) | = 10) | | 8) | 7.71 (d, J = 16) | 8.14 (d, J = 6) | 7.46 (d, J = 2) | salt | |
| | | | | 4.44 (d, J | | | 7.55 (s) | | 7.31 (dd, J = 2, | | |
| | | | | = 10) | | | 6.70 (d, J = 16) | | 8) | | |
| 13 | 3.74 (d, J = 12) | 5.15 (d, J = | 5.70 (dd, J = | 4.36-4.31 | 4.05- | 9.34 (d, J = | 7.86 (s) | 7.83 (s) | 7.80 (d, J = 2) | Bis | MH⁺ = 752 |
| | other | 5) | 5,8) | (m) | 3.95 | 8) | 7.56 (s) | 2.75 (s) | 7.68 (d, J = 2) | zwitterion | |
| | obscured | | | | (m) | | | | 4.40-4.31 (m) | | |
| | | | | | | | | | 3.77 (s) | | |
| | | | | | | | | | 2.66 (s) | | |
| 14 | 3.75 (d, J = 18) | 5.14 (d, J = | 5.68 (dd, J = | 4.40 (d, J | 4.02 (s) | 9.35 (d, J = | 7.87 (s) | 8.89 (d, J = 7) | 4.45 (br s) | Mono | MH⁺ = 710 |
| | 3.47 (d, J = 18) | 5) | 5,8) | = 14) | | 8) | 7.71 (s) | 8.01 (d, J = 7) | 3.80 (br s) | chloride | |
| | | | | 4.34 (d, J | | | 4.61 (s) | | | salt | |
| | | | | = 14) | | | | | | | |
| 15 | 3.50-3.20 (m) | 5.13 (d, J = | 5.66 (dd, J = | 4.40-4.25 | 4.08 (s) | 9.35 (d, J = | 7.88 (s) | 7.81 (s) | 7.76 (d, J = 2) | Bis | MH⁺ = 829 |
| | | 5) | 5,8) | (m) | | 8) | 7.68 (s) | 2.73 (s) | 7.66 (d, J = 2) | chloride | |
| | | | | | | | 4.59 (s) | | 4.65-4.50 (m) | salt | |
| | | | | | | | | | 3.80-3.60 (m) | | |
| | | | | | | | | | 2.70-2.50 (m) | | |
| | | | | | | | | | 2.48 (s) | | |
| | | | | | | | | | 2.30-2.10 (m) | | |
| 16 | 3.69 (d, J = 16) | 5.12 (d, J = | 5.64 (dd, J = | 4.41 (d, J | 3.96 (br | 9.28 (d, J = | 8.83 (t, J = 8) | 8.67 (d, J = 8) | 4.51-4.45 (m) | Mono | MH⁺ = 688 |
| | 3.48 (d, J = 16) | 6) | 6,8) | = 13) | s) | 8) | 7.50 (s) | 8.08 (d, J = 8) | 3.83-3.77 (m) | chloride | |
| | | | | 4.35 (d, J | | | 7.47 (s) | | | salt | |
| | | | | = 13) | | | 3.88 (d, J = 8) | | | | |
| 17 | 3.72 (d, J = 17) | 5.12 (d, J = | 5.66 (dd, J = | 4.41 (d, J | 3.96 (br | 9.30 (d, J = 8) | 8.42 (t, J = 6) | 8.68 (d, J = 8) | 4.48-4.42 (m) | Mono | MH⁺ = 714 |
| | 3.49 (d, J = 17) | 6) | 6,8) | = 11) | s) | | 7.81 (s) | 8.02 (d, J = 8) | 3.81-3.75 (m) | chloride | |
| | | | | 4.34 (d, J | | | 7.56 (d, J = 16) | | | salt | |
| | | | | = 11) | | | 7.55 (s) | | | | |
| | | | | | | | 6.81 (d, J = 16) | | | | |
| | | | | | | | 3.86 (d, J = 6) | | | | |
| 18 | 3.76 (d, J = 17) | 5.15 (d, J = | 5.66 (dd, J = | 4.42 (d, J | 4.03- | 9.31 (d, J = | 7.86 (s) | 8.66 (d, J = 7) | 4.53-4.45 (m) | Mono | M⁺ = 696 |
| | 3.51 (d, J = 17) | 5) | 5,8) | = 12) | 3.93 | 8) | 7.61 (s) | 8.02 (d, J = 7) | 3.83-3.76 (m) | chloride | |
| | | | | 4.35 (d, J | (m) | | 7.35 (d, J = 3) | | | salt | |
| | | | | = 12) | | | 7.24 (d, J = 3) | | | | |
| 19 | 3.74 (d, J = 16) | 5.14 (4, J = | 5.70 (dd, J = | 4.39-4.29 | 3.98 (s) | 9.31 (d, J = | 7.82 (s) | 7.80 (s) | 7.79 (d, J = 1) | Bis | M⁺ = 832 |
| | 3.49 (d, J = 16) | 5) | 5,8) | (m) | | 8) | 7.73 (d, J = 3) | 2.74 (s) | 7.66 (d, J = 1) | chloride | |
| | | | | | | | 7.63 (s) | | 4.46-4.35 (m) | salt | |
| | | | | | | | 7.51 (d, J = 3) | | 4.38-4.31 (m) | | |
| | | | | | | | | | 3.76 (s) | | |
| | | | | | | | | | 2.64 (s) | | |
| | | | | | | | | | 2.30-2.18 (m) | | |
| 20 | 3.45 (d, J = 17) | 4.85 (d, J = | 5.41 (dd, J = | 4.42 (d, J | obscured | 9.22 (d, J = | 7.81 (s) | 7.81 (s) | | mono Na | M⁺ = 712 |
| | 3.26 (d, J = 17) | 5) | 5,8) | = 14) | | 8) | 7.53 (d, J = 16) | 2.61 (s) | | salt/ | |
| | | | | 4.22 (d, J | | | 7.49 (s) | | | zwitterion | |
| | | | | = 14) | | | 6.83 (d, J = 16) | | | | |
| 21 | 3.95-3.87 (m) | 4.86 (d, J = | 5.40 (dd, J = | 4.48 (d, J | 3.92 (br | 9.19 (d, J = | 8.10 (s) | 7.83 (s) | 4.37-4.25 (m) | bis | M⁺ = 837 |
| | | 5) | 5,8) | = 13) | s) | 8) | 7.52 (s) | 2.79 (s) | 3.95-3.85 (m) | zwitterion | |
| | | | | 4.34 (d, J | | | 7.46 (d, J = 16) | | 3.65-3.60 (m) | | |
| | | | | = 13) | | | 7.03 (d, J = 16) | | 3.45-3.15 (m) | | |
| | | | | | | | | | 2.75 (s) | | |
| | | | | | | | | | 2.25-2.15 (m) | | |
| 22 | 3.48 (d, J = 18) | 5.12 (d, J = | 5.68 (dd, J = | 4.40-4.25 | 4.06- | 9.36 (d, J = | 8.35 (t, J = 4) | 7.80 (s) | 7.76 (d, J = 2) | Bis | MH⁺ = 847 |
| | 3.20 (d, J = 18) | 5) | 5,8) | (m) | 3.92 | 8) | 7.87 (s) | 2.74 (s) | 7.61 (d, J = 2) | chloride | |
| | | | | | (m) | | 7.60 (s) | | 4.65-4.50 (m) | salt | |
| | | | | | | | 3.70 (d, J = 4) | | 3.80-3.66 (m) | | |
| | | | | | | | | | 2.72 (s) | | |
| | | | | | | | | | 2.65 (s) | | |
| | | | | | | | | | 2.14-2.07 (m) | | |

We claim:

1. A compound of the formula 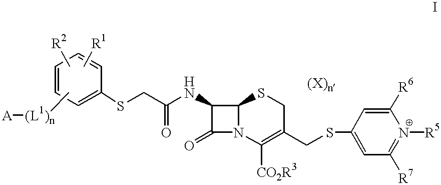
wherein R¹ is hydrogen or halogen; R² is halogen; R³ is hydrogen or a negative charge; A is CO₂R⁴, PO₃(R⁴)₂, SO₃R⁴ or tetrazolyl; R⁴ at each occurrence is either hydrogen or a negative charge; L¹ is a furanyl group, a thienyl group, a C₂-C₁₀ alkylene group, or a C₂-C₁₀ alkylene group wherein one or more of the carbon atoms of said C₂-C₁₀ alkylene may be replaced by S, SO, SO₂, SO₂NH, C(O)NH or NHC(O) and wherein there may be one or more double bonds between adjacent carbon atoms; n is 0 or 1; R⁵ is selected from the group consisting of hydrogen, NH₂, pyrrolidinyl, N═CHR¹ , C₃-C₆ cycloalkyl, C₁-C₆ alkyl, substituted C₂-C₆ alkyl, phenyl, and substituted phenyl, wherein said substituted C₂-C₆ alkyl is a C₂-C₆ alkyl substituted by one or more substituents each independently selected from the group consisting of OH, NR⁸R⁹, NR⁸R⁹R^{9′}, 4-morpholinyl, 4-morpholinyl quaternized by a C₁-C₆ alkyl group, oxo, halogen, CO₂R⁴, SO₃R⁴, PO₃(R⁴)₂, 3-imidazolium, 3-imidazolium substituted by one to two C₁-C₆ alkyl groups, tetrazolyl, and tetrazolyl substituted by one to two C₁-C₆ alkyl groups, and wherein said substituted phenyl is a phenyl substituted by one to three substituents each independently selected from the group consisting of OH, NR⁸R⁹, CO₂R⁴, SO₃R⁴, PO₃(R⁴)₂, 4-morpholinyl, 4-morpholinyl quaternized by a C₁-C₆ alkyl, imidazolyl, imidazolyl substituted by one to two C₁-C₆ alkyl groups, tetrazolyl, and tetrazolyl substituted by one to two C₁-C₆ alkyl groups; R⁶ and R⁷ are each independently hydrogen or C₁-C₆ alkyl; R⁸, R⁹ and R^{9′} are each independently hydrogen or C₁-C₆ alkyl; R¹⁰ is furanyl or thienyl wherein said furanyl or thienyl is optionally substituted by —CO₂R⁴ or —SO₃R⁴; X at each occurrence is a counterion; and n′ is 0 to 2.

2. A compound of claim 1 wherein R¹ is halogen.

3. A compound of claim 2 wherein R¹ and R² are chloro.

4. A compound of claim 3 wherein 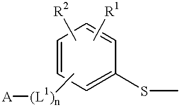
is selected from the group consisting of:

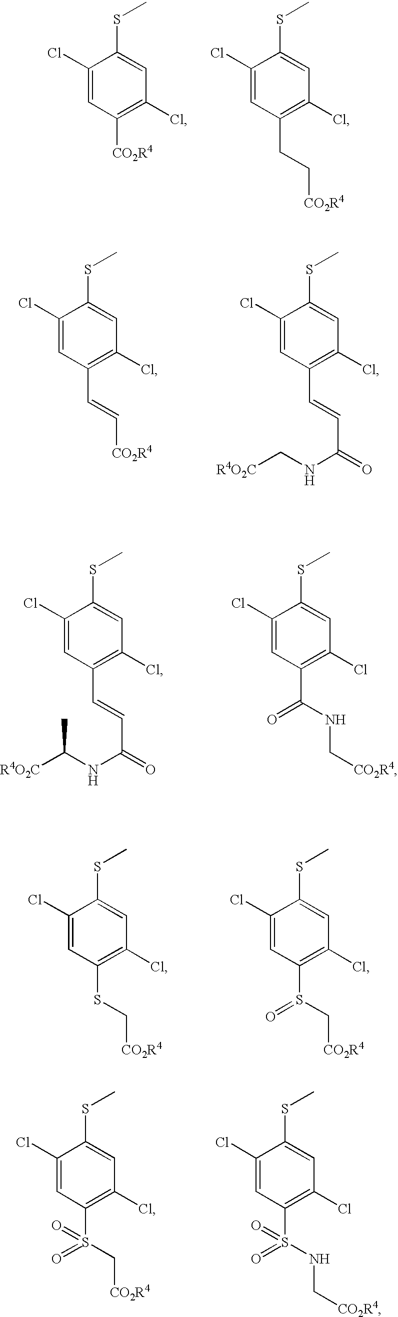

5. A compound of any one of claims 1-4 wherein R⁶ and R⁷ are each independently hydrogen or methyl.

6. A compound of claim 5 wherein 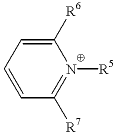
is selected from the group consisting of:

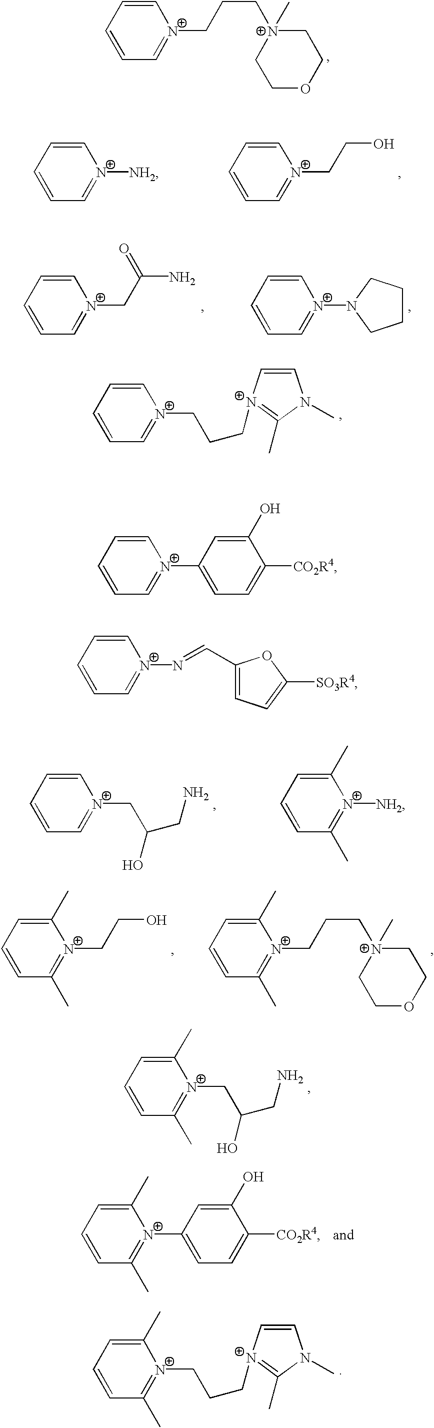

7. A compound of claim 5 wherein R⁶ and R⁷ are hydrogen.

8. A compound of claim 7 wherein 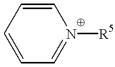
is selected from the group consisting of:

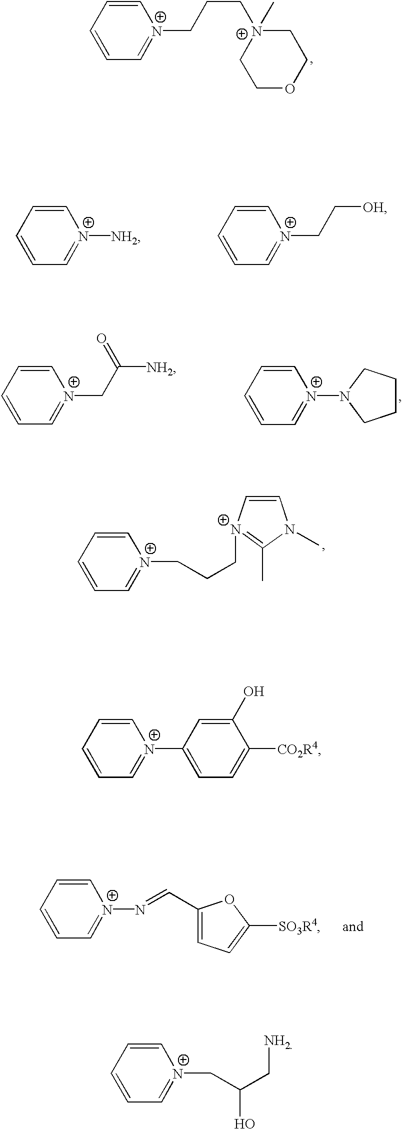

9. A compound of claim 5 wherein R⁶ and R⁷ are methyl.

10. A compound of claim 9 wherein 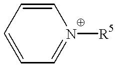
is selected from the group consisting of:

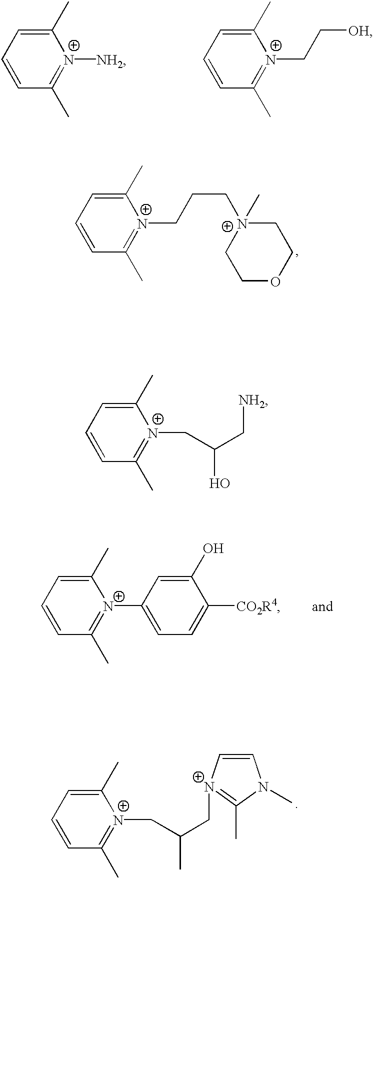

11. A compound of the formula 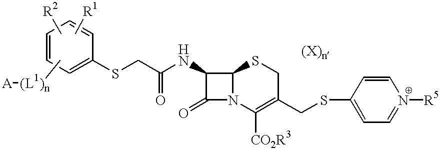
wherein R³ is hydrogen or a negative charge, X at each occurrence is a counterion, n′ is 0 to 2, R⁴ at each occurrence is hydrogen or a negative charge, 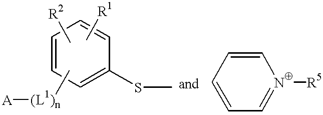
are selected from the group consisting of (a) through (n) as defined below:

| | 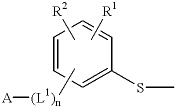 | 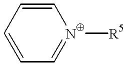 |
|---|---|---|
| (a) | 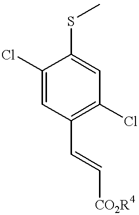 | 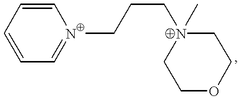 |
| (b) | 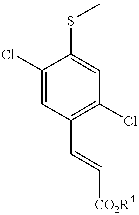 | 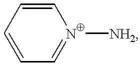 |
| (c) | 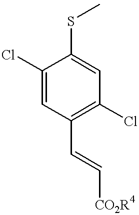 | 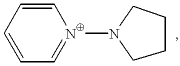 |
| (d) | 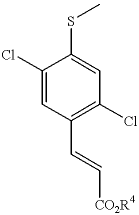 | 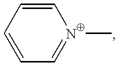 |
| (e) | 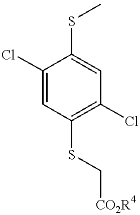 | 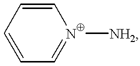 |
| (f) | 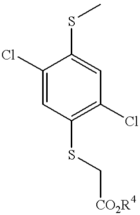 | 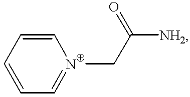 |
| (g) | 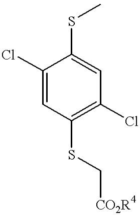 | 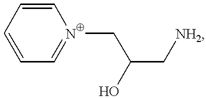 |
| (h) | 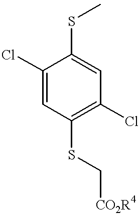 | 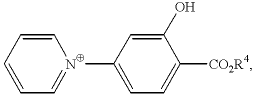 |
| (i) | 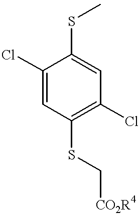 | 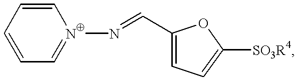 |
| (j) | 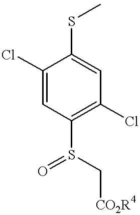 | 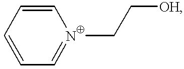 |
| (k) | 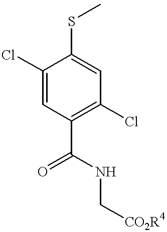 | 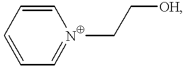 |
| (l) | 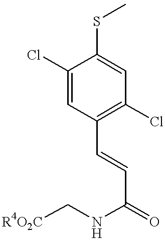 | 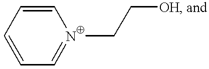 |
| (m) | 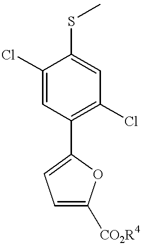 | 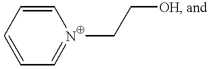 |
| (n) | 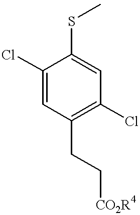 | 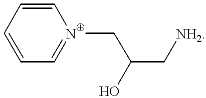 |

12. A compound of the formula 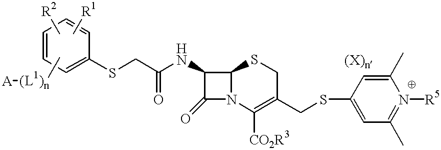
wherein 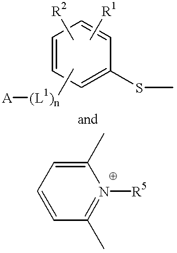
are selected from the group consisting of (a) through (h) as defined below:

| | 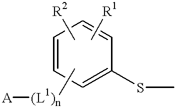 | 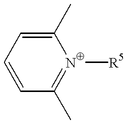 |
|---|---|---|
| (a) | 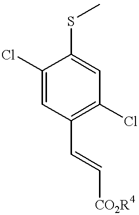 | 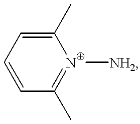 |
| (b) | 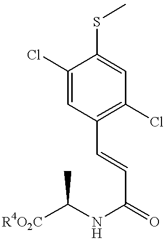 | 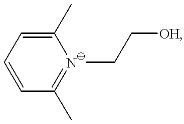 |
| (c) | 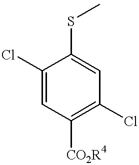 | 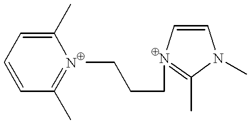 |
| (d) | 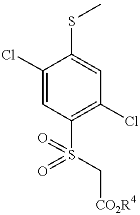 | 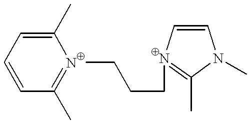 |
| (e) | 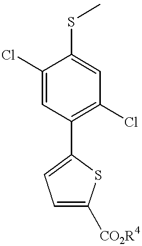 | 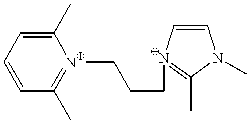 |
| (f) | 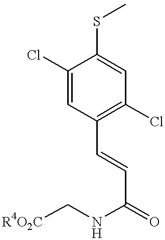 | 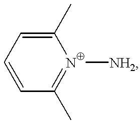 |
| (g) | 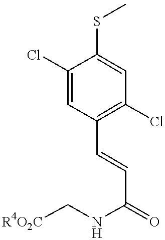 | 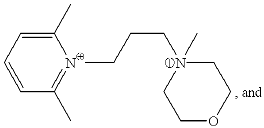 |
| (h) | 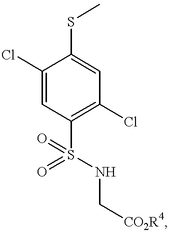 | 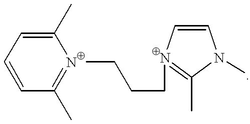 |

13. A compound of the formula 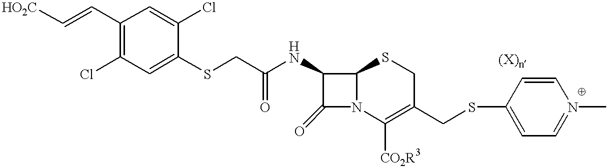
wherein R³ is hydrogen or a negative charge, X is a counter anion, and n′ is 0 or 1, provided that when R³ is hydrogen then n′ is 1, and further provided that when R³ is a negative charge then n′ is 0.

14. The compound of claim 13 wherein R³ is hydrogen, X is chloride and n′ is 1.

15. A compound of the formula 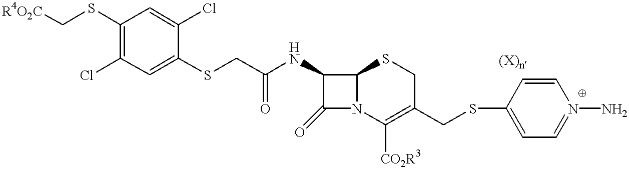
wherein R³ is a negative charge, R⁴ is hydrogen or a negative charge, X is a counter cation, and n′ is 0 or 1, provided that when R⁴ is hydrogen then n′ is 0, and further provided that when R⁴ is a negative charge then n′ is 1.

16. The compound of claim 15 wherein R⁴ is a negative charge, X is a sodium cation and n′ is 1.

17. A compound of the formula 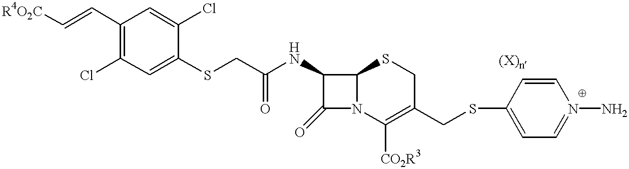
wherein R³ is a negative charge, R⁴ is hydrogen or a negative charge, X is a counter cation, and n′ is 0 or 1, provided that when R⁴ is hydrogen then n′ is 0, and further provided that when R⁴ is a negative charge then n′ is 1.

18. The compound of claim 17 wherein R⁴ is a negative charge, X is a sodium cation and n′ is 1.

19. A compound of the formula 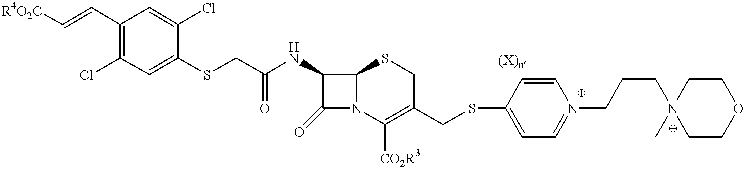
wherein R³ is hydrogen or a negative charge, R⁴ is hydrogen or a negative charge, X is a counter anion, and n′ is 0 or 2, provided that when R³ and R⁴ are hydrogen then n′ is 2, and further provided that when R³ and R⁴ are a negative charge then n′ is 0.

20. The compound of claim 19 wherein R³and R⁴ are a negative charge and n′ is 0.

21. A compound of the formula 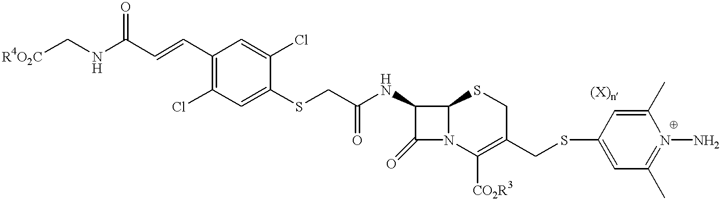
wherein R³ is a negative charge, R⁴ is hydrogen or a negative charge, X is a counter cation, and n′ is 0 or 1, provided that when R⁴ is hydrogen then n′ is 0, and further provided that when R⁴ is a negative charge then n′ is 1.

22. The compound of claim 21 wherein R⁴ is a negative charge, X is a sodium cation and n′ is 1.

23. A compound of the formula 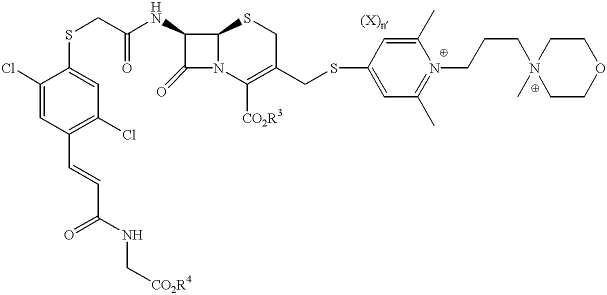
wherein R³ is hydrogen or a negative charge, R⁴ is hydrogen or a negative charge, X is a counter anion, and n′ is 0 or 2, provided when n′ is 2 then R³ and R⁴ are hydrogen, and further provided that when n′ is 0 then R³ and R⁴ are a negative charge.

24. The compound of claim 23 wherein R³ is a negative charge, R⁴ is a negative charge, and n′ is 0.

25. A pharmaceutical composition comprising an effective antibacterial amount of a compound of claim 1 and a pharmaceutically acceptable carrier or excipient.

26. A method of treating a bacterial infection which comprises administering to a host afflicted with such an infection an effective antibacterial amount of a compound of claim 1.

27. A method of treating a bacterial infection caused by a strain of methicillin-resistant Staphylococcus aureus which comprises administering to a host afflicted with such infection an effective antibacterial amount of a compound of claim 1.
